(19) United States
(12) Patent Application Publication
(10) Pub. No.: US 2017/0215846 A1
(43) Pub. Date: Aug. 3, 2017

(54) Medical Devices comprising curved Piezoelectric Transducers

(71) Applicants: THE REGENTS OF THE UNIVERSITY OF CALIFORNIA, Oakland, CA (US); MASDAR INSTITUTE OF SCIENCE AND TECHNOLOGY, Masdar City, Abu Dhabi (AE)

(72) Inventors: Firas SAMMOURA, San Jose, CA (US); Sina AKHBARI, Berkeley, CA (US); Liwei LIN, San Ramon, CA (US)

(21) Appl. No.: 15/112,964

(22) PCT Filed: Jan. 16, 2015
(86) PCT No.: PCT/US15/11847

§ 371 Date: Jul. 20, 2016

Related U.S. Application Data
(60) Provisional application No. 61/931,493, filed on Jan. 24, 2014.

(51) Int. Cl. A61B 8/00; H01L 41/053; H01L 41/332; B06B 1/06; A61B 8/08; A61N 7/00; G10K 9/125; G10K 11/28; H01L 41/09; A61B 8/06

(52) U.S. Cl. CPC A61B 8/4494; H01L 41/098; H01L 41/053; H01L 41/332; A61B 8/06; A61B 8/0858; A61B 8/0808; A61B 8/0875; A61B 8/0891; A61N 7/00; G10K 9/125; G10K 11/28; B06B 1/0666

(57) ABSTRACT

Medical devices configured to direct sound waves to a body tissue of a subject are provided. The medical device includes a housing and a curved piezoelectric transducer, where the curved piezoelectric transducer is configured to direct sound waves produced by the curved piezoelectric transducer to the body tissue of the subject. Also provided are methods of directing sound waves to a body tissue of a subject using the subject medical devices. The subject medical devices and methods find use in a variety of applications where the treatment of a body tissue of a subject with sound waves is desired.

### CROSS-REFERENCE TO RELATED APPLICATION

Pursuant to 35 U.S.C. §119(e), this application claims priority to the filing date of U.S. Provisional Application Ser. No. 61/931,493 filed on Jan. 24, 2014, the disclosure of which is incorporated herein by reference.

### INTRODUCTION

Ultrasonic imaging is one of the most important and widely used medical imaging techniques, which uses high-frequency sound waves to take images of soft tissues, such as muscles, internal organs, as well as blood flows in blood vessels. Medical sonography (ultrasonography) can be used to capture their size, structure and any pathological lesions with real time tomographic images. Ultrasound is also used to visualize fetuses during routine and emergency prenatal care, e.g., obstetric sonography.

Many different types of images can be formed using ultrasound, such as a B-mode image, which displays the acoustic impedance of a two-dimensional cross-section of tissue. To obtain an ultrasound image, a sound wave is produced by a transducer. Electrical pulses from the ultrasound device can be used to drive the transducer at the desired frequency. The sound waves travel into the body and focus at a desired depth. The sound wave is partially reflected from the layers between different tissues or scattered from smaller structures, for example sound is reflected where there may be acoustic impedance changes in the body, e.g. blood cells in blood plasma, small structures in organs, etc. Some of the reflected sound waves return to the transducer, where they are detected and transformed into an image based on how long it took the reflected sound waves to be received from when the sound was transmitted, and the intensity of the reflected sound waves.

### SUMMARY

Medical devices configured to direct sound waves to a body tissue of a subject are provided. The medical device includes a housing and a curved piezoelectric transducer, where the curved piezoelectric transducer is configured to direct sound waves produced by the curved piezoelectric transducer to the body tissue of the subject. Also provided are methods of directing sound waves to a body tissue of a subject using the subject medical devices. The subject medical devices and methods find use in a variety of applications where the treatment of a body tissue of a subject with sound waves is desired.

Aspects of the present disclosure include a medical device configured to direct sound waves to a body tissue of a subject. The medical device includes a housing, and a curved piezoelectric transducer. The curved piezoelectric transducer includes a substrate, a curved support layer having a peripheral portion in contact with the substrate, and a curved piezoelectric element disposed on the curved support layer. The curved piezoelectric transducer is configured to direct sound waves produced by the curved piezoelectric transducer to the body tissue of the subject.

In some embodiments, the medical device includes a processor connected to the curved piezoelectric transducer.

In some embodiments, the medical device includes a sound wave detector connected to the processor.

In some embodiments, the medical device includes a wireless communication device connected to the processor.

In some embodiments, the medical device is selected from an imaging device, a therapeutic device, and a measurement device.

In some embodiments, the sound waves are ultrasound waves.

In some embodiments, the curved piezoelectric element includes a first electrode layer, a piezoelectric layer, and a second electrode layer.

Aspects of the present disclosure include a method of directing sound waves to a body tissue of a subject. The method includes producing sound waves from a medical device, where the medical device includes a housing and a curved piezoelectric transducer. The curved piezoelectric transducer includes a substrate, a curved support layer having a peripheral portion in contact with the substrate, and a curved piezoelectric element disposed on the curved support layer, where the curved piezoelectric transducer is configured to direct the produced sound waves to the body tissue of the subject.

In some embodiments, the method is a method of imaging the body tissue of the subject. In some embodiments, the method includes applying the produced sound waves to the body tissue from the curved piezoelectric transducer, detecting sound waves reflected from the body tissue, and producing an image of the body tissue from the detected sound waves.

In some embodiments, the body tissue is brain tissue, muscle, bone, tendon, ligament, fat, blood vessel, skin, and connective tissue.

In some embodiments, the method is a method of treating the body tissue of the subject with therapeutic sound waves. In some embodiments, the method includes applying the produced sound waves to the body tissue of the subject from the curved piezoelectric transducer to treat the subject for a condition in need of treatment.

In some embodiments, the condition is selected from stroke, myocardial infarction, soft tissue injury, tendon injury, kidney stones, varicose veins, and cellulite.

In some embodiments, the condition is selected from ligament sprain, muscle strain, tendonitis, joint inflammation, plantar fasciitis, metatarsalgia, facet irritation, impingement syndrome, bursitis, rheumatoid arthritis, osteoarthritis, scar tissue adhesion, gallstones, cataracts, and tumor.

In some embodiments, the method is a method of measuring a parameter of a body tissue or fluid of the subject. In some embodiments, the method includes applying the produced sound waves to the body tissue or fluid from the curved piezoelectric transducer to measure the parameter of the body tissue or fluid.

In some embodiments, the parameter is a thickness of the body tissue, a density of the body tissue or fluid, or a velocity of a flow of the fluid.

In some embodiments, the sound waves are ultrasound waves.

In some embodiments, the curved piezoelectric element includes a first electrode layer, a piezoelectric layer, and a second electrode layer.

### BRIEF DESCRIPTION OF THE DRAWINGS

FIG. 1 shows a three dimensional cross-section providing an example of a curved pMUT of the present disclosure.

FIG. 2a shows a schematic diagram of a curved pMUT, according to embodiments of the present disclosure.

FIG. 2b shows a schematic diagram of a planar pMUT structure.

FIG. 2c shows a cross-section view of the curved of FIG. 1.

FIG. 2d shows a three-dimensional view of portions of the curved pMUT structure in contact with the underlying substrate (i.e., the “clamp sections”), according to embodiments of the present disclosure.

FIG. 2e shows embodiments of possible variations of the clamped section, according to embodiments of the present disclosure.

FIG. 3a and FIG. 3b show cross-sectional views of the motion of a curved pMUT, according to embodiments of the present disclosure.

FIG. 4a and FIG. 4b show graphs of displacement versus frequency of a curved pMUT, according to embodiments of the present disclosure.

FIG. 5 shows a scanning electron microscope (SEM) image of a cross-section of a curved pMUT, according to embodiments of the present disclosure.

FIG. 6 shows an SEM image showing the polarization of the aluminum nitride layer, according to embodiments of the present disclosure.

FIG. 7 shows an SEM image showing the polarization direction on a curved pMUT, according to embodiments of the present disclosure.

FIG. 8 shows computer simulation results for a curved pMUT total displacement, according to embodiments of the present disclosure.

FIG. 9a and FIG. 9b show cross-sectional drawings of a curved pMUT with different curvatures, according to embodiments of the present disclosure.

FIGS. 10a-10d show a flow diagram showing a fabrication process for a curved pMUT, according to embodiments of the present disclosure.

FIG. 11 shows a graph of a comparison between simulation and experimental results for center displacement (nm/V) and resonant frequency (MHz) vs. radius of curvature (μm), according to embodiments of the present disclosure.

FIGS. 12a-12f show a flow diagram of a thermal deformation fabrication process for an array of curved pMUT devices, according to embodiments of the present disclosure.

FIGS. 13a-13d show a flow diagram of a curved pMUT fabrication process, according to embodiments of the present disclosure.

FIG. 14 shows a fabrication process using a mandrel, according to embodiments of the present disclosure.

FIG. 15 shows a curved pMUT array with an offset providing an angled transmission, according to embodiments of the present disclosure.

FIGS. 16a-16e show a curved pMUT fabrication process where silicon bending is used to fabricate curves, according to embodiments of the present disclosure.

FIG. 17 shows a curved pMUT with a constant radius (top) and a curved pMUT with a double curvature of radius (bottom), according to embodiments of the present disclosure.

FIG. 18a and FIG. 18b show the motion of a curved pMUT in both transmit and receive modes, according to embodiments of the present disclosure.

FIG. 19a shows a graph of the normalized radial displacement of the curved pMUT vs. tangential angular position φ (deg), and FIG. 19b shows a graph of center displacement nm/V vs. frequency (Hz) for a curved pMUT, according to embodiments of the present disclosure.

FIG. 20 shows a 2-dimensional schematic of the axisymmetric curved pMUT with a clamped boundary condition, according to embodiments of the present disclosure.

FIG. 21 shows graphs of theoretical results of input impedance versus frequency of a curved pMUT, according to embodiments of the present disclosure.

FIG. 22 shows a graph of impedance phase (Ω) versus frequency (MHz) measurements (in air) of a curved pMUT, according to embodiments of the present disclosure.

FIG. 23 shows a graph of center displacement (nm) vs. input voltage, Vₚₚ, (V) of a curved pMUT, according to embodiments of the present disclosure.

FIG. 24 shows schematics of equivalent circuit models of the curved pMUT, according to embodiments of the present disclosure.

FIG. 25 shows a schematic of equivalent circuit models for operation of the curved pMUT in a vacuum, according to embodiments of the present disclosure.

FIG. 26 shows a 3D cross-sectional view of a stress engineered self-curved pMUT fabricated in a CMOS-compatible process, according to embodiments of the present disclosure.

FIG. 27 (top) shows a cross-sectional view of a concave shape curved pMUT fabricated by a stress engineering process due to the SiN and low temperature oxide (LTO) films with tensile and compressive residual stresses, respectively, according to embodiments of the present disclosure. FIG. 27 (bottom) shows a cross-sectional view after adding the bottom and top electrodes and the AlN layer to complete the stress engineered curved pMUT fabrication.

FIG. 28 shows a process flow diagram for the stress-engineered curved pMUT, according to embodiments of the present disclosure. FIG. 28, panel a, shows a silicon nitride deposition and patterning. FIG. 28, panel b, shows LTO deposition and chemical mechanical polishing (CMP). FIG. 28, panel c, shows backside deep reactive ion etching (DRIE) to form the concave-shape diaphragm. FIG. 28, panel d, shows Mo/AlN/Mo sputtering and via opening to the bottom electrode.

FIG. 29 shows confocal laser scanned images of a fabricated curved pMUT, according to embodiments of the present disclosure: FIG. 29, panel a, top view; FIG. 29, panel b, measured curvature profile; and FIG. 29, panel c, 3D tilted view and the radius of curvature.

FIG. 30, panel a, and FIG. 30, panel b, show tilted and front view SEM micrographs of two self-curved pMUTs after the devices were cleaved, according to embodiments of the present disclosure. FIG. 30, panel c, shows a released diaphragm showing the stack of the pMUT layers, and FIG. 30, panel d, shows an enlarged view showing good crystal alignment of AlN on the curved diaphragm.

FIG. 31 shows a graph of center deflection versus nitride radial coverage (%) for devices with 200 μm in nominal radii using a 650 nm-thick nitride layer, according to embodiments of the present disclosure. Results showed good consistency among simulation, theory, and experimental data.

FIG. 32 shows a graph of measured dynamic responses of stress-engineered curved pMUTs without (released) and with (unreleased) the bottom silicon layer, according to embodiments of the present disclosure. The pMUTs had 200 μm in average radius and 2.7 μm center deflection before release. The AlN, Si, and BOX layer thicknesses were 2 μm, 4 μm, and 1 μm, respectively.

FIG. 33 shows a graph of simulated dynamic responses of a stressed engineered curved pMUT with 200 μm in average radius and 2.34 μm center diaphragm displacement for −50, 0, 50, 100, and 150 MPa residual stress in the AlN layer, according to embodiments of the present disclosure.

### DETAILED DESCRIPTION

Medical devices configured to direct sound waves to a body tissue of a subject are provided. The medical device includes a housing and a curved piezoelectric transducer, where the curved piezoelectric transducer is configured to direct sound waves produced by the curved piezoelectric transducer to the body tissue of the subject. Also provided are methods of directing sound waves to a body tissue of a subject using the subject medical devices. The subject medical devices and methods find use in a variety of applications where the treatment of a body tissue of a subject with sound waves is desired.

Medical devices that include a curved piezoelectric transducer or an array of curved piezoelectric transducers as described herein may vary and can include any such medical device where a curved piezoelectric transducer or an array of curved piezoelectric transducers finds use. Examples of medical devices that may include a curved piezoelectric transducer or an array of curved piezoelectric transducers include, but are not limited to, ultrasound medical devices. In general, a “medical device” is an instrument, machine, apparatus, or similar or related article that is used for the diagnosis of disease or other conditions, or in the cure, mitigation, treatment, or prevention of disease, in humans or other animals. In some instances, medical devices do not achieve their primary purpose through chemical action within or on the body of the subject and do not depend on metabolic action to achieve their primary purpose.

Embodiments of the medical devices described herein are configured to direct sound waves to a body tissue of a subject. In some instances, the sound waves are ultrasound waves. As such, in certain cases, the medical device is an ultrasound medical device. Various types of medical devices and uses thereof are described herein.

The section below describes curved piezoelectric transducers in more detail, and is followed by a description of devices, e.g., medical devices, that include such curved piezoelectric transducers. Methods of using the subject medical devices and methods of making the curved piezoelectric transducers used in such medical devices are described in more detail in the subsequent sections.

### Curved Piezoelectric Transducers

Aspects of the present disclosure include medical devices having one or more curved piezoelectric transducers. Embodiments of the curved piezoelectric transducer include a substrate, a curved support layer having a peripheral portion in contact with the substrate, and a curved piezoelectric element disposed on the curved support layer. Methods of making the curved piezoelectric transducers are also provided. The curved piezoelectric transducers, devices and methods find use in a variety of applications, including devices, such as electronics devices, having one or more (e.g., an array) of the curved piezoelectric transducers on a substrate.

Curved piezoelectric transducers (e.g., curved piezoelectric micromachined ultrasonic transducers or curved pMUTs) are provided. In some instances, the curved piezoelectric transducer is produced using a complementary metal-oxide semiconductor (CMOS)-compatible fabrication process. Curved piezoelectric transducers of the present disclosure find use in a variety of applications, e.g., where ultrasonic transducers are desired that have high coupling and acoustic pressure, and higher DC displacements, as compared with planar pMUTs of similar geometry. In some instances, curved pMUTs described herein are based on a CMOS-compatible fabrication process using CMOS-compatible materials, such as, but not limited to, aluminum nitride (AlN), as the transduction material. Micro-fabrication techniques may be used to control the radius of curvature of working pMUTs, e.g., from 400 μm to 2000 μm.

Curved pMUTs of the present disclosure may provide one or more of the following: an increase in bandwidth, flexible transducer geometries, natural acoustic impedance matched with water, reduced voltage requirements, mixing of different resonant frequencies, and facilitated integration with electronic circuits, such as circuits for miniaturized high frequency applications. Curved pMUTs of the present disclosure may also be provided in a pMUT array format, which finds use in a variety of applications, such as, but not limited to, gesture recognition and fingerprint ID systems. Curved pMUTs also find use in sensor systems, facilitating practical and reasonable cost incorporation into various consumer electronic products.

Aspects of the present disclosure include a curved piezoelectric transducer. As used herein, a curved piezoelectric transducer may also be referred to as a curved piezoelectric micromachined ultrasonic transducer, or curved pMUT, a “membrane”, or a “diaphragm”. In certain embodiments, the curved piezoelectric transducer is provided on a substrate. The substrate may be any convenient substrate that is compatible with the curved piezoelectric transducer and materials therein, as well as the fabrication process for the curved piezoelectric transducer. For example, the substrate may be composed of a material that is compatible with integrated circuits and fabrication processes for integrated circuits. In some instances, the substrate is compatible with complementary metal-oxide semiconductor (CMOS) fabrication processes. For example, the substrate may be composed of a material compatible with deposition processes, such as chemical and/or physical layer deposition processes, etching, lithography, combinations thereof, and the like. In certain embodiments, the substrate is a semiconductor material, such as, but not limited to, silicon, silicon nitride, combinations thereof, and the like. In certain embodiments, the substrate is silicon.

In certain embodiments, the curved piezoelectric transducer is disposed on the substrate. For instance, the curved piezoelectric transducer may be provided on a surface of the substrate, such as on a top surface of the substrate. At least a portion of the curved piezoelectric transducer may be in contact with the surface of the substrate. For example, as described in more detail below, a peripheral portion of the curved piezoelectric transducer may be in contact with the substrate, while a central portion of the curved piezoelectric transducer does not contact the substrate. The central portion of the curved piezoelectric transducer that is not in contact with the substrate may be exposed through an opening in the substrate. The opening in the substrate (also referred to herein as a “via” or a “hole”) may extend through the entire thickness of the substrate such that a portion of the surface of the curved piezoelectric transducer, e.g., a bottom surface of the curved piezoelectric transducer, is exposed. By “exposed” is meant that the surface is in contact with the surrounding environment and does not substantially contact the underlying substrate. In certain instances, the opening through the substrate may be cylindrical in shape and may have an average diameter ranging from 10 μm to 5 mm, such as from 10 μm to 2 mm, or 20 μm to 1 mm, or 30 μm to 500 μm, or 40 μm to 200 μm, or 50 μm to 200 μm, or 100 μm to 200 μm, or 120 μm to 180 μm. In other embodiments, the central portion of the curved piezoelectric transducer that is not in contact with the substrate may be suspended over the substrate. In these embodiments, the central portion that is not in contact with the underlying substrate may be suspended over the substrate by the peripheral portion of the curved piezoelectric transducer, which is disposed on the substrate. One or more layers may be provided between the peripheral portion of the curved piezoelectric transducer and the substrate to elevate the central portion of the curved piezoelectric transducer above the surface of the substrate.

In certain embodiments, the curved piezoelectric transducer includes a support layer, where at least a portion of the support layer is non-planar. In some cases, the curved piezoelectric transducer includes a curved support layer. The curved support layer may include a portion that has a curved shape, thus providing the curved piezoelectric transducer with a curved shape. In some instances, the curved piezoelectric transducer includes a curved support layer, where the curved portion of the support layer is either convex or concave in shape. In certain cases, the curved piezoelectric transducer includes a curved support layer, where a portion of the curved support layer is concave in shape (e.g., has a curvature similar to a depression in the substrate). For instance, a curved support layer having a concave shape may have a portion (e.g., a central portion) that extends towards or below the surface of the substrate. In certain cases, the curved piezoelectric transducer includes a curved support layer, where a portion of the curved support layer is convex in shape (e.g., has a curvature similar to a hump in the substrate). For instance, a curved support layer having a convex shape may have a portion (e.g., a central portion) that extends away from or above the surface of the substrate. In some instances, the curved support layer (and thus the curved piezoelectric transducer) is curved in a spherical shape or a portion os a spherical shape (e.g., hemispherical shape).

In certain embodiments, the curved piezoelectric transducer can have a radius of curvature of from 10 μm to 10,000 μm, such as from 20 μm to 8000 μm, including 50 μm to 5000 μm, or 100 μm to 2000 μm, or 500 μm to 1500 μm, or 600 μm to 1000 μm. In some instances, the curved piezoelectric transducer is circular in shape. The average diameter of such curved piezoelectric transducers can be from 10 μm to 5 mm, such as from 10 μm to 2 mm, or 20 μm to 1 mm, or 30 μm to 500 μm, or 40 μm to 200 μm, or 50 μm to 200 μm, or 100 μm to 200 μm, or 120 μm to 180 μm. In certain embodiments, the curved support layer of the curved piezoelectric transducer has a radius of curvature substantially the same as that of the curved piezoelectric transducer described above. In certain embodiments, the curved support layer is circular in shape and has a diameter that is substantially the same as that of the curved piezoelectric transducer described above. In some instances, the curved support layer has an average thickness ranging from 100 nm to 10 μm, such as from 250 nm to 10 μm, or 500 nm to 10 μm, or 750 nm to 10 μm, or 1 μm to 10 μm, or 1 μm to 9 μm, or 1 μm to 8 μm, or 1 μm to 7 μm, or 1 μm to 6 μm, or from 2 μm to 6 μm, or 3 μm to 6 μm, or 4 μm to 6 μm. In some cases, the curved support layer has an average thickness of 5 μm. As used in the present disclosure, the term “average” refers to the arithmetic mean. Average thickness refers to a layer, where the layer may have a thickness that varies from one region of the layer to another region of the layer; the average thickness is the average of the various thicknesses of the regions of the layer.

In some embodiments, the curved support layer includes a central portion and a peripheral portion. The peripheral portion of the support layer may surround the periphery of the central portion. For example, the peripheral portion may be adjacent to and in contact with the external edges of the central portion of the support layer. In certain cases, the central portion of the support layer is circular in shape. In some embodiments, a circular central portion of the support layer has an diameter ranging from 1 μm to 1 mm, such as from 1 μm to 750 μm, or 1 μm to 500 μm, or 1 μm to 250 μm, or 5 μm to 200 μm, or 10 μm to 200 μm, or 10 μm to 150 μm. In certain embodiments, the central portion of the support layer is surrounded by the peripheral portion as described above. In embodiments where the central portion is circular in shape, the surrounding peripheral portion may have an annular (i.e., ring) shape. In some cases, the circular central portion and annular peripheral portion are concentric. In some instances, the central portion is partially surrounded by the peripheral portion. For example, the peripheral portion may surround a segment of the central portion that is less than the entire periphery of the central portion, such as 99% or less, or 97% or less, or 95% or less, or 90% or less, or 85% or less, or 80% or less, or 75% or less, or 70% or less, or 65% or less, or 60% or less, or 55% or less, or 50% or less.

In some embodiments, the central portion and peripheral portion are formed of the same material. In these embodiments, the support layer may be substantially contiguous, such that there are no boundaries between the central portion and the peripheral portion of the support layer. In other embodiments, the central portion and the peripheral portion of the support layer may be formed of different materials. In these cases, the peripheral portion may surround the periphery of the central portion, where the materials of the central portion and peripheral portion are in contact with each other along substantially the entire periphery of the central portion. As such, in embodiments where the central portion and peripheral portion of the support layer are composed of different materials, they may still form a contiguous support layer where there are substantially no gaps or discontinuities between the central portion and peripheral portion of the support layer.

In certain embodiments, the curved support layer of the curved piezoelectric transducer may include a portion in contact with the substrate. In these embodiments, a portion of the support layer may not be in contact with the substrate. In certain embodiments, the central portion of the curved support layer has a curved shape as described herein, and at least a portion of the curved central portion of the support layer may not be in contact with the substrate. In these embodiments, the curved central portion of the support layer that is not in contact with the substrate may facilitate movement of the curved central portion of the support layer when the curved piezoelectric transducer is in use. In certain embodiments, at least part of the peripheral portion of the support layer is in contact with the substrate. For example, a part of the peripheral portion of the support layer may support the support layer on the substrate. In some instances, the peripheral portion of the support layer may contact the substrate and suspend the central portion, which is not in contact with the substrate as described above, over the substrate. In these embodiments, the curved piezoelectric transducer may be supported on the substrate by the peripheral portion of the support layer while allowing the curved central portion of the support layer to move when the curved piezoelectric transducer is in use.

In certain embodiments, as described above, the curved piezoelectric transducer includes a curved support layer, where the curved support layer has a convex and/or concave shape. In some instances, the curved piezoelectric transducer includes a curved support layer, where a portion of the curved support layer has a concave shape and a portion of the curved support layer has a convex shape. For example, the central portion of the support layer may have a concave shape as described above. In some instances, the peripheral portion of the support layer has a convex shape. As such, in these embodiments, the support layer may have a concave-convex structure (also referred to herein as a hybrid concave-convex structure), where the central portion has a concave shape and the peripheral portion has a convex shape.

The support layer may be composed of any convenient material that is compatible with the curved piezoelectric transducer and other materials therein, as well as the fabrication process for the curved piezoelectric transducer. For example, the support layer may be a material that is compatible with integrated circuits and fabrication processes for integrated circuits. In some instances, the support layer is compatible with complementary metal-oxide semiconductor (CMOS) fabrication processes. For example, the support layer may be composed of a material compatible with deposition processes, such as chemical and/or physical layer deposition processes, etching, lithography, combinations thereof, and the like. In certain embodiments, the support layer is a semiconductor material, such as, but not limited to, silicon, silicon nitride, combinations thereof, and the like. In some cases, the support layer is composed of an oxide, such as, but not limited to, a low temperature oxide, e.g., silicon dioxide, and the like. As described above, in some instances, the support layer is a contiguous support layer, and as such may be composed of a substantially homogeneous material, such as materials described above. In other embodiments, as described above, the support layer may include portions composed of different materials (e.g., a central portion and a peripheral portion composed of different materials). In these embodiments, the different portions may be composed of any of the different materials described herein. For example, the central portion of the support layer may be composed of a semiconductor material as described herein, such as, but not limited to, silicon, silicon nitride, combinations thereof, and the like. In some cases, the peripheral portion of the support layer may be composed of a different material from the central portion, such as, but not limited to, an oxide, e.g. a low temperature oxide, such as silicon dioxide, and the like. In other embodiments, the peripheral portion of the support layer may be composed of a semiconductor material as described herein, such as, but not limited to, silicon, silicon nitride, combinations thereof, and the like. In other cases, the central portion of the support layer may be composed of a different material from the peripheral portion, such as, but not limited to, an oxide, e.g. a low temperature oxide, such as silicon dioxide, and the like.

In certain embodiments, the support layer may be composed of a single support layer or two or more sub-support layers. For example, the support layer may be a single support layer as described above. In other embodiments, the support layer is composed of two or more sub-support layers, where the multiple sub-support layers are disposed one on top of another. Each sub-support layer may be composed of the same or different material, e.g., any of the support layer materials as described herein. For instance, the curved piezoelectric transducer may include a first sub-support layer and a second sub-support layer. The first sub-support layer may be composed of any of the support materials described herein, such as, for example, an oxide, e.g. a low temperature oxide, such as silicon dioxide, and the like. Disposed on a surface of the first sub-support layer may be a second sub-support layer. In some instances, the second sub-support layer may be composed of the same material as the first sub-support layer. In other embodiments, the second sub-support layer is composed of a different material than the first sub-support layer. For example, the second sub-support layer may be composed of a semiconductor material as described herein, such as, but not limited to, silicon, silicon nitride, combinations thereof, and the like. In some cases, the second sub-support layer is composed of silicon. Additional sub-support layers may be provided on a surface of the second sub-support layer. For example, a third sub-support layer may be disposed on a surface of the second sub-support layer. The third sub-support layer may be composed of the same material as the first sub-support layer and/or the second sub-support layer. In other embodiments, the third sub-support layer is composed of a different material than the first sub-support layer and/or the second sub-support layer. For example, the third sub-support layer may be composed of a combination of different materials, such as a central portion composed of a semiconductor material as described herein (e.g., silicon, silicon nitride, combinations thereof, and the like), and a peripheral portion composed of a different material than the central portion, such as an oxide, e.g. a low temperature oxide, such as silicon dioxide, and the like. In some embodiments, a sub-support layer that is composed of a combination of different materials may include a central portion composed of silicon nitride and a peripheral portion composed of silicon dioxide.

In certain embodiments, the curved piezoelectric transducer includes a curved piezoelectric element disposed on the curved support layer. The curved piezoelectric element may be disposed on a surface of the curved support layer, such as on a surface of the curved support layer opposite the exposed surface of the curved support layer. In some instances, the curved piezoelectric element includes several layers, which together compose the curved piezoelectric element. In some cases, the curved piezoelectric element includes one or more electrode layers, and a piezoelectric layer. The one or more electrode layers may be disposed on opposing surfaces of the piezoelectric layer. For example, the piezoelectric element may include a first electrode layer, a piezoelectric layer, and a second electrode layer. The first electrode layer may be disposed on the support layer, the piezoelectric layer may be disposed on the first electrode layer, and the second electrode layer may be disposed on the piezoelectric layer. Each layer of the piezoelectric element may be curved, having approximately the same radius of curvature as the curved piezoelectric transducer and curved support layer, as described herein.

The piezoelectric element may be composed of any convenient material. For example, the electrode layers may be composed of an electrically conductive material, such as, but not limited to, a metal (e.g., molybdenum (Mo), copper (Cu), silver (Ag), gold (Au), platinum (Pt), combinations thereof, and the like. In some instances, the electrode layer is composed of molybdenum. In some instances, the electrode layer is composed of platinum. In certain embodiments, the piezoelectric layer is composed of a piezoelectric material, such as, but not limited to, the following: a piezoelectric ceramic, e.g., barium titanate (BaTiO₃), lead zirconate titanate (Pb[ZrₓTi₁₋ₓ]O₃, where 0≦x≦1; PZT), potassium niobate (KNbO₃), lithium niobate (LiNbO₃, lithium tantalate (LiTaO₃), sodium tungstate (Na₂WO₃), Ba₂NaNb₅O₅, Pb₂KNb₅O₁₅, zinc oxide (ZnO), sodium niobate (NaNbO₃), potassium niobate (KNbO₃), bismuth ferrite (BiFeO₃), bismuth titanate Bi₄Ti₃O₁₂, sodium bismuth titanate Na_{0.5}Bi_{0.5}TiO₃, or combinations thereof; a piezoelectric semiconductor, e.g., GaN, InN, AlN, ZnO, or combinations thereof; or a polymer, e.g., polyvinylidene fluoride (PVDF); or combinations thereof, and the like. In certain embodiments, the piezoelectric layer is composed of AlN. In certain embodiments, the piezoelectric layer is composed of PZT.

In certain embodiments, the electrode layers of the piezoelectric element have a thickness ranging from 1 nm to 1000 nm, such as 5 nm to 900 nm, or 10 nm to 800 nm, or 25 nm to 700 nm, or 50 nm to 600 nm, or 50 nm to 500 nm, or 50 nm to 400 nm, or 50 nm to 300 nm, or 50 nm to 200 nm, or 100 nm to 200 nm. In some cases, the electrode layers of the piezoelectric element have a thickness ranging from 100 nm to 200 nm, such as 150 nm. In certain embodiments, the piezoelectric layer of the piezoelectric element has a thickness ranging from 100 nm to 10 μm, such as 250 nm to 9 μm, or 500 nm to 8 μm, or 750 nm to 7 μm, or 1 μm to 6 μm, or 1 μm to 5 μm, or 1 μm to 4 μm, or 1 μm to 3 μm. In some cases, the piezoelectric layer of the piezoelectric element has a thickness ranging from 1 μm to 3 μm, such as 2 μm.

### Energy & Power Consumption

Embodiments of the curved piezoelectric transducer (curved pMUT) find use, e.g., in battery-powered devices, which may require low power dissipation. In some instances, the curved pMUT can have a lower power consumption as compared to typical piezoelectric transducers. By example, a curved pMUT of the present disclosure, in comparison to a typical flat pMUT (i.e., planar pMUT) of the same diameter, uses 1× to 100× less power, such as 10× to 50× less power, for instance 20× less power. In certain embodiments, a curved pMUT consumes 0.01 mJ to 0.1 mJ of energy or less when activated, such as 10 μJ to 100 μJ or less, or 5 μJ to 75 μJ or less, or 1 μJ to 50 μJ or less, or 0.1 μJ to 50 μJ or less. The power consumption of the curved piezoelectric transducer (or an array containing a plurality of piezoelectric transducers) may vary depending on the application and desired features of the device. For example, a fingerprint sensor that includes a curved pMUT array of the present disclosure may have different power consumptions depending on the resolution, such as, 500 dpi or 300 dpi (e.g., with or without phased array beam forming) and fabrication technology. In some cases, the energy consumption of a curved pMUT array for a single fingerprint scan is about 1 μJ to 40 μJ, such as 5 μJ to 30 μJ, or 10 μJ to 20 μJ, which may be significantly lower energy consumption as compared to a typical planar pMUT. Similar differences in power consumption may be present for other applications, such as curved pMUT or curved pMUT arrays used for gesture recognition. In certain embodiments, the curved pMUT uses an AC drive voltage for activation of the curved pMUT. The AC drive voltage used to power the curved pMUT may range from 0.1 V to 50 V, such as 0.1 V to 25 V, or 0.5 V to 10 V, or 1 V to 5 V, including 2 V to 3 V.

Energy consumption of the curved piezoelectric transducer may also depend on the frequency of use of the curved piezoelectric transducer. For example, the frequency at which a fingerprint sensor is used may depend on the application, e.g., fingerprint sensors used in smart phones may be used each time the device is activated by the user, typically a few times per hour or day. High security applications may use frequent re-verification, for example each minute, which increases the frequency of use of the curved piezoelectric transducer. Door locks equipped with fingerprint sensors, e.g., for access to residential homes or automobiles, may be used with less frequency, such as a few times per day.

In certain embodiments, the curved piezoelectric transducer (e.g., the curved pMUT array fingerprint sensor) is configured to be activated only when used to facilitate a minimization in energy consumption. Activation of the curved piezoelectric transducer can be controlled, for example with software, by a capacitive sensor, or the curved pMUT array itself. For instance, control of activation by the curved pMUT itself may be achieved by having a single or small number of curved pMUTs in an array activated periodically, for example ten times per second. Since only a few curved pMUTs out of the entire array are periodically activated, the power dissipation of this operation may be lower than if the entire array was periodically activated (e.g., 0.01 μW or less, depending on the design). If a finger or other object is detected, the entire curved pMUT array can be activated to acquire a fingerprint pattern. The resulting low average power dissipation of the curved pMUT fingerprint sensor can facilitate use of the curved pMUT as a replacement for a power switch in certain applications, such as smart phones; the device is turned on only when a valid fingerprint is recognized with no other steps needed. This mode of operation can facilitate the convenience and security provided to the user.

The energy stored in a CR2032 lithium coin cell battery is typically 2000 to 3000 Joules, which allows for tens to hundreds of millions of finger print recognitions. If, for example, the fingerprint sensor is used once per hour, the coin cell battery may last over 400 years if used only for powering the fingerprint sensor. Since smart phone batteries have an order-of-magnitude higher energy capacity than a typical coin cell battery, the addition of a fingerprint sensor with a curved pMUT array to such a device would result in negligible reduction of the running time per battery charge.

An example of the power dissipation of a device employing a curved pMUT array is described below. The actual power dissipation may deviate from this estimate because of variations in the design. A curved pMUT array fingerprint sensor may have a total area of 1 cm by 2 cm. Assuming 500 dpi resolution, this sensor may include an array of 200 by 400 individual curved pMUTs.

Energy consumption during a transmit phase may be due to charging and discharging the capacitance of the curved pMUTs and the electrical wiring. Although this capacitance may depend on details of the fabrication technology, the capacitance per curved pMUT may typically be 1 pF or less. Activating all curved pMUTs with 10V for 4 cycles thus consumes 1.6 μJ of energy. Depending on requirements of the application, all curved pMUT transmitters can be activated at once, or sequentially, or a combination thereof. Energy consumption may be independent of the activation protocol used. In a phased array mode, the energy consumption may be higher since several (e.g., 10 or more, such as 20 or more, for example 21) curved pMUTs may be activated to sense a single point.

The energy consumption for reception includes the energy needed for amplifying the signal and the energy needed for analog-to-digital conversion of the signal. Since the receiver may be active for only a short period after an acoustic pulse has been transmitted, energy consumption can be reduced by power gating. For example, an acoustic signal traveling 300 μm to 750 μm from the transducer to the dermis and back at a typical sound velocity of 1500 m/s experiences a 200 ns to 500 ns delay during most of which the receiving amplifier is ready to accept and amplify the echo signal. Assuming 1 mW average power dissipation for an amplifier with approximately 1 GHz bandwidth, the energy required to process the echo signals at all 200 by 400 curved pMUTs is 40 μJ. An 8-bit analog-to-digital converter operating at 100 MHz to convert the echo amplitudes to digital signals consumes a similar amount of energy. In summary, the total energy consumption to transmit, receive, and digitize the acoustic signals in a 1 cm by 2 cm curved pMUT array may be equal to about 1.6 μJ+4 μJ+40 μJ or about 46 μJ if no beam forming is used. With beam forming, the energy may be one to two orders of magnitude larger, depending on the number of curved pMUTs activated per beam. Additional energy may be used to process, identify, and validate fingerprints acquired by the curved pMUT array. The level of energy consumption may depend on the processor and the complexity of the algorithms used and for efficient realizations is typically 1 mJ or less.

### Post-Processing Tuning

In certain embodiments, a curved pMUT can facilitate correction of drift and manufacturing errors. When a curved pMUT has residual stress, it may change the initial deflection, without breaking the device. Afterwards, the change in the initial deflection can be corrected by tuning the device with circuitry. Thus, such defects are correctable in the curved pMUT system as compared to typical planar pMUTs, where such changes in deflection may not be correctable. Curved pMUTs of the present disclosure thus facilitate correction of process-related issues with circuits.

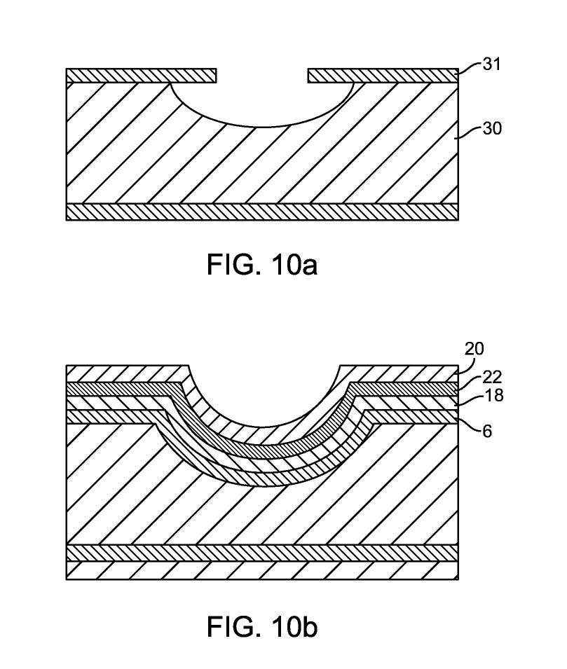

The change of curvature induced in curved pMUTs by stress is also described in FIG. 11. As shown in FIG. 11, a change of curvature induced in curved pMUTs by stress can be used by purposely applying stress to the curved pMUTs via a DC bias. Such application of stress may change the radius of curvature in a predictable manner. In some instances, each curved pMUT can be tuned to a desired frequency of operation. Such tuning can be performed after fabrication, compensating for manufacturing errors. This aspect of the curved pMUTs may increase the final yield and quality of a fabrication process, providing substantial cost savings, lower cost, and higher quality products.

Active tuning of the curved pMUTs finds use in arrays of pMUTs. Each pMUT within an array may be tuned at one particular frequency. If there are manufacturing errors among the curved pMUTs, application of a small amount of DC bias can be utilized to change the radius of curvature in a precise, incremental manner, thus compensating for any manufacturing errors. This post-processing tuning method may facilitate fabrication of curved pMUT arrays, for example for fingerprint ID systems and motion sensors. Active tuning of the curved pMUT may facilitate an increase in the production level of functioning devices. This aspect may also facilitate pMUT array device fabrication because each pMUT may be matched to the same frequency by post-processing tuning. The post-process tuning aspect of curved pMUTs facilitates lower cost, higher yield product production, which in turn makes the curved pMUT easier to engineer and to fabricate. Being able to tune all the curved pMUTs with circuits allows tuning to be separated from the fabrication process, which lessens or eliminates the time and effort needed to tune and re-tune the fabrication process.

### Electromechanical Coupling

Curved pMUTs of the present disclosure may have higher levels of electromechanical coupling as compared to typical planar pMUTs. The electromechanical coupling of the curved pMUT may depend on the medium through which the signal is transmitted, and also the material from which the curved pMUT is fabricated. This provides flexibility in designing curved pMUTs to meet the particular needs of a sensing system. By example, choice of fabrication materials allows balancing costs of materials, ease of manufacture, and performance in design criteria to meet requirements of a final sensing system for specific applications.

In certain embodiments, a curved pMUT electromechanical coupling performance ranges from 0.1% to 100%, such as from 1% to 100%, or from 5% to 100%, or 10% to 100%, or 10% to 90%, or 10% to 80%, or 10% to 70%, to 10% to 60%, or 10% to 50%, or 20% to 50%, or 25% to 45%, or 30% to 45%. For example, a curved pMUT electromechanical coupling performance in air when fabricated with aluminum nitride may range from 0.1% to 100%, such as 0.1% to 75%, or 0.1% to 50%, or 0.1% to 25%, or 0.1% to 10%, or 0.1% to 5%, or 0.2% to 4.8%, or 1% to 3%, such as 2%. In other instances, a curved pMUT electromechanical coupling performance in air when fabricated with lead zirconium titanate (PZT) may range from 10% to 50%, such as 20% to 40%, e.g., 30%. In other embodiments, a curved pMUT electromechanical coupling performance in air when fabricated with lead magnesium niobate-lead titanate (PMN-PT) may range from 45% to 100%, such as 50% to 98%, e.g., 90% or 92%. Other materials than those specified in the above examples, as well as alloys or amalgams of two or more of those materials, may be used in designing a specific curved pMUT to provide the desired pMUT characteristics for a specific application.

### Immunity to Residual Stress

Curved pMUTs of the present disclosure may have significant immunity to residual stress. By immunity to residual stress in meant that a curved pMUT may be subjected to residual stress of a certain value or range without a significant degradation in the performance of the curved pMUT. A curved pMUT subject to residual stress releases the stress to adopt a curved configuration having minimal residual stress. In contrast, a planar pMUT has no room for release of residual stress. Analogous to a guitar string, a curved pMUT may deflect less with more tension. As a result, a curved pMUT may have significant immunity to residual stress. In certain instances, the residual stress will cause a change to the initial deflection, and change the curvature of the curved pMUT to relieve the residual stress. In other words, residual stress may be relieved by changing the curvature of the pMUT structure. In the analyses presented here, stress-free curved pMUTs are used in the analyses, assuming the residual stress effects are substantially dissipated to produce substantially stress-free pMUTs. For example, FIG. 11 shows a graph of initial deflection (e.g., center displacement, nm/V) and resonant frequency (MHz) with respect to various radii of curvature (μm), assuming the residual stress effect has been transferred to changes in the radius of curvature. As shown in FIG. 11, the frequency changed with the radius of curvature.

The curved pMUT resistance to residual stress may vary depending on the materials used, which allows for design of curved pMUTs having a desired immunity to residual stress depending on the material used. By example, the curved pMUT immunity to residual stress in aluminum ranges from 10 MPa to 500 MPa, such as 50 MPa to 400 MPa, or 100 MPa to 300 MPa.

### Responsivity

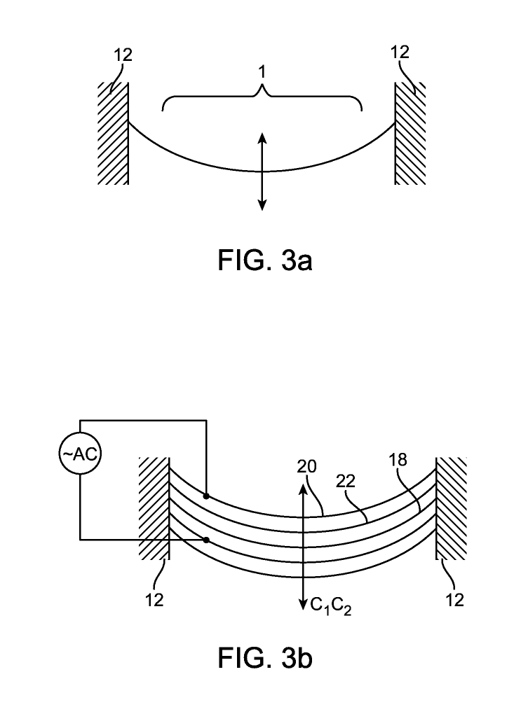

As described in more detail regarding FIG. 4a and FIG. 4b, a curved pMUT may have a significantly higher responsivity as compared to a typical planar pMUT. The DC response/displacement achieved by a curved pMUT may depend on the material used, but generally, a curved pMUT of the present disclosure has a DC response from 0.1 nm/V to 100 nm/V, such as 0.1 nm/V to 75 nm/V, or 0.1 nm/V to 50 nm/V, or 0.1 nm/V to 25 nm/V, or 0.5 nm/V to 20 nm/V, or 0.5 nm/V to 10 nm/V, or 0.5 nm/V to 5 nm/V, for instance 1 nm/V. The curved pMUT response as compared to typical planar devices of the same average diameter shows an increase in responsivity from 10× to 100×, such as 20× to 70×, for example 50×.

### Curved Piezoelectric Transducer Devices

Aspects of the present disclosure include devices that have one or more curved piezoelectric transducers as disclosed herein. In certain embodiments, the curved piezoelectric transducers may be arranged as an array of curved piezoelectric transducers. For instance, an array of curved piezoelectric transducers may be provided on a substrate, where the substrate can be a substrate as described herein.

An “array” includes any two-dimensional or substantially two-dimensional (as well as a three-dimensional) arrangement of curved piezoelectric transducers. In some instances, the curved piezoelectric transducers form addressable regions, e.g., spatially addressable regions. An array is “addressable” when it has multiple curved piezoelectric transducers positioned at particular predetermined locations (e.g., “addresses”) on the array. Array features (e.g., curved piezoelectric transducers) may be separated by intervening spaces. Any given substrate may carry 1, 2, 3, 4, 5, 6, 7, 8, 9, 10, or more arrays disposed on a surface of the substrate. Depending upon the use, any or all of the arrays may be the same or different from one another and each may contain multiple distinct curved piezoelectric transducers. An array may contain one or more, including two or more, four or more, 8 or more, 10 or more, 50 or more, 100 or more, 150 or more, 200 or more, 250 or more, 300 or more, 350 or more, 400 or more, 450 or more, 500 or more, 550 or more, 600 or more, 650 or more, 700 or more, 750 or more, 800 or more, 850 or more, 900 or more, 950 or more, 1000 or more, 1250 or more, 1500 or more, 2000 or more, 2500 or more, 3000 or more, 4000 or more, 5000 or more, 6000 or more, 7000 or more, 8000 or more, 9000 or more, or 10,000 or more curved piezoelectric transducers. In certain embodiments, the curved piezoelectric transducers can be arranged into an array with an area of less than 10 cm², or less than 5 cm², e.g., less than 1 cm², including less than 50 mm², less than 20 mm², such as less than 10 mm², or even smaller. For example, curved piezoelectric transducers may have dimensions in the range of 10 μm to 5 mm, such as from 10 μm to 2 mm, or 20 μm to 1 mm, or 30 μm to 500 μm, or 40 μm to 200 μm, or 50 μm to 200 μm, or 100 μm to 200 μm, or 120 μm to 180 μm.

In certain embodiments, the device that includes a curved piezoelectric transducer or an array of curved piezoelectric transducers is a portable device. For example, the device that includes a curved piezoelectric transducer or an array of curved piezoelectric transducers may be a hand-held device (e.g., a device that may be held and operated by a single hand or by two hands of a user). In some instances, the device that includes a curved piezoelectric transducer or an array of curved piezoelectric transducers is a battery operated device. A battery operated device may be powered from one or more batteries contained in the device or electrically connected to the device. In some cases, a battery operated device does not require a connection to a power outlet to have sufficient power to operate.

### Medical Devices

Devices that include a curved piezoelectric transducer or an array of curved piezoelectric transducers may vary and can include any such device where a curved piezoelectric transducer or an array of curved piezoelectric transducers finds use. Examples of devices that may include a curved piezoelectric transducer or an array of curved piezoelectric transducers include, but are not limited to, medical devices. In certain embodiments, the medical device is configured to direct sound waves to a body tissue of a subject. In some instances, the sound waves are ultrasound waves. By “ultrasound” is meant that the sound waves have a frequency greater than the upper limit of the human hearing range. For example, ultrasound may have a frequency of 20 kHz or more, such as 50 kHz or more, or 100 kHz or more, or 250 kHz or more, or 500 kHz or more, or 750 kHz or more, or 1 MHz or more, or 10 MHz, or more, or 25 MHz or more, or 50 MHz or more, or 100 MHz or more, or 250 MHz or more, or 500 MHz or more, or 750 MHz or more, or 1 GHz or more, or 5 GHz or more, or 10 GHz or more, or 25 GHz or more, or 50 GHz or more, or 75 GHz or more, or 100 GHz or more. In certain instances, a subject medical device produces ultrasound with a frequency ranging from 200 kHz to 100 MHz, such as 200 kHz to 75 MHz, or 250 kHz to 50 MHz, or 250 kHz to 25 MHz, or 250 kHz to 10 MHz.

In certain embodiments, a medical device of the present disclosure includes a housing and a curved piezoelectric transducer as described herein. As described above, the device may be a portable device, and as such the housing may be sized to be portable. For example, the housing may be a hand-held housing (e.g., a housing that may be held and operated by a single hand or by two hands of a user). The housing may contain the curved piezoelectric transducer (or array or curved piezoelectric transducers). In some embodiments, the housing may also contain additional components of the medical device, such as, but not limited to a processor, a sound wave detector, a power source (e.g., battery), a wireless communication device, a data storage device, a user interface device, combinations thereof, and the like. In some instances, the housing has dimensions of 250 cm or less (e.g., a longest dimension, such as height, width or thickness), such as 240 cm or less, or 230 cm or less, or 220 cm or less, or 210 cm or less, or 200 cm or less, or 190 cm or less, or 180 cm or less, or 170 cm or less, or 160 cm or less, or 150 cm or less, or 140 cm or less, or 130 cm or less, or 120 cm or less, or 110 cm or less, or 100 cm or less, or 90 cm or less, or 80 cm or less, or 70 cm or less, or 60 cm or less, or 50 cm or less, or 40 cm or less, or 30 cm or less, or 20 cm or less, or 10 cm or less.

As described herein, the curved piezoelectric transducer of the medical device may include a substrate, a curved support layer, and a curved piezoelectric element disposed on the curved support layer. The curved piezoelectric transducer may be configured to direct sound waves (e.g., ultrasound) produced by the curved piezoelectric transducer to a body tissue of a subject.

Examples of medical devices that include a subject curved piezoelectric transducer or array of curved piezoelectric transducers include, but are not limited to, imaging devices, therapeutic devices and measurement devices. For example, medical imaging devices may be configured to direct sound waves (e.g., ultrasound) produced by a curved piezoelectric transducer (or array of curved piezoelectric transducers) to a body tissue of a subject to produce an image (e.g., an ultrasound image) of the target body tissue of the subject. In other embodiments, therapeutic medical devices may be configured to direct sound waves (e.g., ultrasound) produced by a curved piezoelectric transducer (or array of curved piezoelectric transducers) to a body tissue of a subject to treat the body tissue of the subject with therapeutic sound waves. In other embodiments, medical measurement devices may be configured to direct sound waves (e.g., ultrasound) produced by a curved piezoelectric transducer (or array of curved piezoelectric transducers) to a body tissue or fluid of a subject to measure a parameter of the body tissue or fluid of the subject.

In certain embodiments, the medical devices are computer-based medical devices. A “computer-based medical device” refers to the hardware, software, and data storage components used to produce and analyze the sound waves from the curved piezoelectric transducer (or array or curved piezoelectric transducers). The hardware of the computer-based medical device may include a central processing unit (CPU), inputs, outputs, and data storage components. The data storage components may include any computer readable medium that includes a device for recording sound waves produced by the curved piezoelectric transducer, or an accessible memory component that can store signals detected from sound waves produced by the curved piezoelectric transducer.

To “record” data, programming or other information on a computer readable medium refers to a process for storing information, using any convenient method. Any convenient data storage structure may be used, depending on the method used to access the stored information. A variety of data processor programs and formats can be used for storage, e.g. word processing text file, database format, etc.

In certain embodiments, the medical device includes a processor. The processor may be configured to operably couple to the curved piezoelectric transducer (or array or curved piezoelectric transducers). In some instances, the processor is electrically coupled to the curved piezoelectric transducer (or array or curved piezoelectric transducers). The processor may be electrically coupled to the curved piezoelectric transducer so as to provide power, activation signals, etc. to components of the medical device, such as, but not limited to the curved piezoelectric transducer and/or arrays thereof. As such, the processor may include an activation signal generator. The activation signal generator may be configured to provide power, activation signals, etc. to components of the medical device, such as, but not limited to the curved piezoelectric transducer and/or arrays thereof.

Additionally, the processor may be configured to analyze signals, such as signals detected from sound waves produced by the curved piezoelectric transducer. Depending on the type of medical device, the signals may be used to produce images of a body tissue, measure a parameter of the body tissue, and the like. In some instances, the processor may include be configured to output a result in response to analyzing the signals. Thus, the processor may be configured to receive detected signals, process the signals, obtain a result from the signals, and output the result to a user in a human-readable or an audible format.

A “processor” references any hardware and/or software combination that will perform one or more programmed functions. For example, any processor herein may be a programmable digital microprocessor such as available in the form of an electronic controller, central processing unit, server or personal computer (e.g., desktop or portable). Where the processor is programmable, suitable programming can be communicated from a remote location to the processor, or previously saved in a computer program product (such as a portable or fixed computer readable storage medium, whether magnetic, optical or solid-state device based). For example, a magnetic medium, optical disk or solid-state memory device may carry the programming, and can be read by a suitable reader communicating with the processor.

In some instances, the subject medical device is configured to modulate the power applied to the curved piezoelectric transducer. Modulating the power applied to the curved piezoelectric transducer may facilitate focusing of the curved piezoelectric transducer, such as dynamic focusing of the curved piezoelectric transducer during use.

Embodiments of the subject systems may also include a wired or wireless communication device configured to transfer information between the medical device and one or more users, e.g., via a user computer, as described below. In certain embodiments, a computer program product is provided that includes a computer-usable medium having control logic (e.g., a computer software program, including program code) stored therein. The control logic, when executed by the processor, causes the processor to perform functions described herein.

In addition to the curved piezoelectric transducer and processor, the medical device may include a number of additional components, such as, but not limited to: data output devices, e.g., monitors, speakers, etc.; data input devices, e.g., interface ports, buttons, switches, keyboards, touchscreens, etc.; power sources, e.g., battery; power amplifiers; wired or wireless communication components; etc.

In certain embodiments, the medical device includes a display. The display may be configured to provide a visual indication of a result obtained from the processor, as described above. For instance, the display may be configured to display a image (e.g., ultrasound image) of a body tissue if the medical device is configured as a medical imaging device. In some embodiments, the display may be configured to display a measurement result to a user, where the medical device is configured as a medical measurement device, e.g., a quantitative or qualitative measurement of a thickness of a body tissue, a density of a body tissue or fluid, a velocity of a flow of a body fluid, and the like.

The medical device optionally includes a programmable memory, which prior to and during the use of the medical device can be programmed with relevant information such as: calibration data for the curved piezoelectric transducer or array thereof; a record of completed assay steps; a record about which sample was imaged, measured or treated; a record of the measurement results; a record of the obtained image; and the like.

### Curved Piezoelectric Transducer Devices

Further examples of devices that may include a curved piezoelectric transducer or an array of curved piezoelectric transducers include, but are not limited to, sensor devices, such as gesture recognition sensors (e.g., gesture recognition sensors in cell phones, tablet computers, personal computers, video game systems, etc.), fingerprint detection sensors (e.g., fingerprint detection sensors in cell phones, tablet computers, personal computers, security systems, etc.), body motion sensors, sensors for measuring liquid and/or gas velocity, sensors for measuring speed through air or water, distance sensors (e.g., automotive sensors for parking assist technology), location sensors (e.g., sonar, underwater range finders, Ultrasound Identification (USID), Real Time Locating System (RTLS), or Indoor Positioning System (IPS)), sensors for detecting uneven surfaces, alarm sensors (e.g., burglar alarm sensors), sensors for liquid measurement (e.g., sensors for liquid tank or channel level measurements), touchless sensing devices (e.g., sensors for non-destructive testing, level sensors or sensing systems that require no contact with the target, etc.), and the like.

Examples of other types of devices that may include a curved piezoelectric transducer or an array of curved piezoelectric transducers include, but are not limited to, ultrasonic transducer devices, e.g., devices that convert energy into ultrasound. Ultrasonic transducer devices can apply the generated ultrasound to a subject or an object. For example, ultrasonic transducer devices include, but are not limited to, ultrasonic impact treatment (UIT) devices (e.g., devices that use ultrasound to enhance the mechanical and/or physical properties of metals), devices for processing of liquids and slurries, ultrasound cleaning devices, humidifiers, defrosters, and the like.

Devices that include a curved piezoelectric transducer or an array of curved piezoelectric transducers may also include devices used for the transmission of data (e.g., CDMA cellphones).

### Methods of Use

Aspects of the present disclosure include methods of using the subject curved piezoelectric transducer. In certain embodiments, the methods of use are performed in the context of directing sound waves produced by the curved piezoelectric transducer to a body tissue of a subject. As such, in certain aspects, methods of the present disclosure include producing sound waves from a medical device that includes a curved piezoelectric transducer. The curved piezoelectric transducer may be configured to direct the produced sound waves from the curved piezoelectric transducer to the body tissue of the subject. As described herein, the medical device may include a housing and a curved piezoelectric transducer, where the curved piezoelectric transducer includes a substrate, a curved support layer, and a curved piezoelectric element disposed on the curved support layer.

In certain embodiments, the method of use is a method of imaging a body tissue of a subject. In some instances, the method of imaging the body tissue includes applying the sound waves produced by the curved piezoelectric transducer from the curved piezoelectric transducer to the body tissue or the subject. The method of imaging the body tissue may further include detecting sound waves reflected from the body tissue of the subject, and producing an image of the body tissue from the detected sound waves. In certain embodiments, the sound waves produced by the curved piezoelectric transducer are ultrasound waves. In these embodiments, the method of imaging the body tissue is a method of ultrasound imaging.

In certain embodiments, detecting the sound waves reflected from the body tissue of the subject includes detecting the sound waves using a sound wave detector. The sound wave detector may be a separate component of the medical device that is operably connected to the processor and configured to detect sound waves and communicate a respective signal to the processor. In some instances, the sound waves are also detected by the curved piezoelectric transducer. In these embodiments, the curved piezoelectric transducer may be configured to produce sound waves and detect sound waves. In certain embodiments, the step of producing an image of the body tissue from the detected sound waves includes analyzing the detected sound waves to produce the image of the body tissue. Analyzing the sound waves may be performed by the processor of the medical device as described herein.

In certain instances, the body tissue that is imaged by the subject methods includes, but is not limited to, brain tissue, muscle, bone, tendon, ligament, fat, blood vessel, skin, connective tissue, combinations thereof, and the like.

Embodiments of the methods of use also include methods of treating a body tissue of a subject with therapeutic sound waves (e.g., therapeutic ultrasound). In certain instances, the method of treatment includes applying sound waves produced by a curved piezoelectric transducer from the curved piezoelectric transducer to the body tissue of the subject to treat the subject for a condition in need of treatment. The term “treating” or “treatment” as used herein means the treating or treatment of a disease or medical condition in a subject, such as a mammal (e.g., a human) and includes: (a) preventing the disease or medical condition from occurring, such as, prophylactic treatment of a subject; (b) ameliorating the disease or medical condition, such as, eliminating or causing regression of the disease or medical condition in a subject; (c) suppressing the disease or medical condition, for example by, slowing or arresting the development of the disease or medical condition in a subject; and/or (d) alleviating a symptom of the disease or medical condition in a subject.

The subject to be treated can be one that is in need of therapy, where the subject to be treated is one amenable to treatment using the therapeutic sound waves (e.g., therapeutic ultrasound). Accordingly, a variety of subjects may be amenable to treatment using the medical devices disclosed herein. Generally, such subjects are “mammals”, with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys, etc.).

In certain embodiments, the condition to be treated can be, but is not limited to, stroke, myocardial infarction, soft tissue injury, tendon injury, kidney stones, varicose veins, or cellulite. In certain embodiments, the condition to be treated can be, but is not limited to, ligament sprain, muscle strain, tendonitis, joint inflammation, plantar fasciitis, metatarsalgia, facet irritation, impingement syndrome, bursitis, rheumatoid arthritis, osteoarthritis, scar tissue adhesion, gallstones, cataracts, or tumor.

For example, ultrasound pulses can be used to break calculi such as kidney stones and gallstones into fragments small enough to be passed from the body. Medical uses may also include cleaning teeth in dental hygiene using ultrasound. Ultrasound medical devices may be used for cataract treatment by phacoemulsification. In addition, ultrasound medical devices may be used to ablate tumors or other tissue non-invasively, e.g., High Intensity Focused Ultrasound (HIFU), also called Focused Ultrasound Surgery (FUS). Methods of use of the subject medical devices may also include enhancing drug uptake using acoustic targeted drug delivery (ATDD), such as delivering chemotherapy to cancer cells or various drugs to other target tissues, or triggering the release of drugs from delivery vectors including liposomes, polymeric microspheres and self-assembled polymeric drug delivery vehicles.

Examples of other medical treatment methods include, but are not limited to, the following: ultrasound-guided sclerotherapy and endovenous laser treatment for the non-surgical treatment of varicose veins; ultrasound-assisted lipectomy (e.g., liposuction assisted by ultrasound); acoustophoresis for contactless separation, concentration and manipulation of microparticles and biological cells; therapeutic tooth and bone regeneration; transcranial ultrasound, e.g., for use in treating stroke; and the like.

Aspects of the methods of using the subject medical devices also include methods of measuring a parameter of a body tissue or fluid of a subject. In some instances methods of measuring a parameter of a body tissue include applying sound waves produced by a curved piezoelectric transducer from the curved piezoelectric transducer to the body tissue or fluid to measure the parameter of the body tissue or fluid. In some cases, the parameter to be measured includes, but is not limited to, a thickness of the body tissue, a density of the body tissue or fluid, a velocity of a flow of the fluid, and the like. In certain embodiments, the sound waves produced by the curved piezoelectric transducer are ultrasound waves.

### Methods of Making

Aspects of the present disclosure further include methods of making a curved piezoelectric transducer. In certain embodiments, the methods of making a curved piezoelectric transducer include producing a curved piezoelectric element on a curved support layer. The curved support layer may be present on a surface of a substrate as described herein. For example, the curved support layer may include a peripheral portion disposed on a surface of the substrate.

In certain embodiments, the methods of making the curved piezoelectric transducer include processes compatible with CMOS fabrication protocols. For example, the methods of making the curved piezoelectric transducer may include one or more processes, such as etching, lithography, physical deposition, chemical deposition, combinations thereof, and the like. Deposition processes as described herein may include any convenient thin film deposition processes, such as, but not limited to, chemical vapor deposition (CVD), physical vapor deposition (PVD), sputtering, combinations thereof, and the like.

In certain aspects, the method of making a curved piezoelectric transducer begins with a substrate as described herein. The curved piezoelectric transducer may be produced on a surface of the substrate. For example, the method may include forming a curved depression in a surface of the substrate. The curved depression may be curved in the same desired shape (e.g., diameter, radius of curvature, etc.) as the resulting curved piezoelectric transducer. Forming the curved depression in the surface of the substrate may include etching (e.g., wet chemical or dry plasma etching) the surface of the substrate to form the curved depression. In some instances, a mask is applied to the surface of the substrate prior to the etching step. The mask may have one or more holes, through which one or more curved depressions may be formed in the surface of the substrate (e.g., by etching the surface of the substrate as described above).

In certain embodiments, the method of making the curved piezoelectric transducer further includes depositing a support layer in the curved depression. The support layer may be any support layer and/or multiple layers of support layers as described herein. In some instances, the mask is removed from the surface of the substrate prior to depositing the support layer in the curved depression. In certain cases, the support layer is deposited in the curved depression or a portion of the curved depression. In some cases, the support layer is deposited in the curved depression and also on the surface of the substrate adjacent to the curved depression. As such, the support layer may have a central portion deposited in the curved depression, and a peripheral portion deposited on the surface of the substrate adjacent to the curved depress. As described above, the central portion and the peripheral portion of the support layer may form a substantially contiguous layer. In certain embodiments, the support layer includes multiple layers. For instance, the bottom layer of the support layer may be composed of an oxide (e.g., SiO₂) and the overlying layer(s) of the support layer may be composed of silicon.

In certain embodiments, the method of making the curved piezoelectric transducer further includes depositing a piezoelectric element on the support layer. As described herein, a piezoelectric element may include multiple layers, such as a first electrode layer, a piezoelectric layer, and a second electrode layer. The layers of the piezoelectric element may be deposited on the support layer to form the piezoelectric element on a surface of the support layer.

In some instances, the method of making the curved piezoelectric transducer further includes removing substrate material from an opposing surface of the substrate. The opposing surface may be a surface opposite from the substrate surface where the support layer and piezoelectric element are deposited. In some instances, removing substrate material from the opposing surface of the substrate produces an opening (also referred to as a via or a hole herein) through the substrate. As described herein, the opening through the substrate may expose a portion of the curved support layer. For instance, the exposed surface of the support layer may be the surface of the support layer opposite from the surface where the piezoelectric element is deposited.

In certain embodiments, the method of making the curved piezoelectric transducer further includes forming an electrical contact to the first electrode layer of the piezoelectric element, and forming an electrical contact to the second electrode layer of the piezoelectric element. To form an electrical contact to the first electrode layer, the method may also include removing a portion of the overlying piezoelectric layer and the overlying second electrode layer to form a hole or via exposing a portion of the surface of the first electrode layer. In some instances, removing a portion of the overlying piezoelectric layer and the overlying second electrode layer includes etching hole or via to expose a portion of the surface of the first electrode layer.

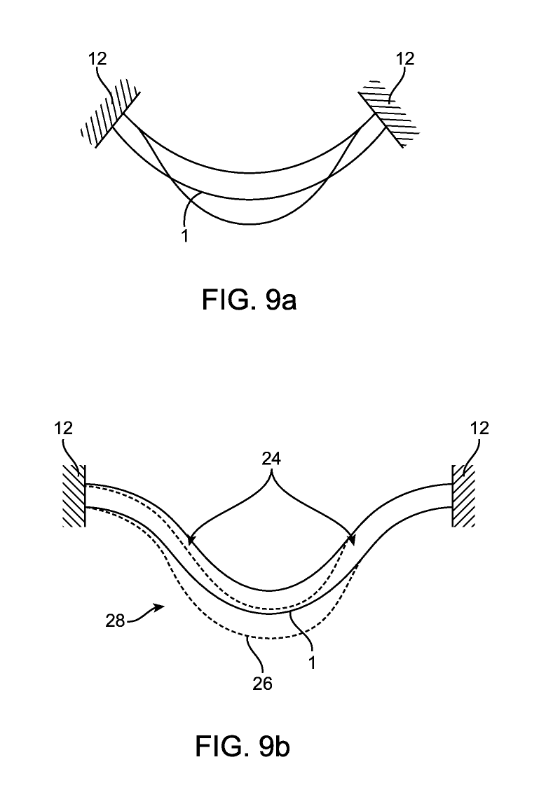

FIGS. 10a-10d show a flow diagram of a fabrication process for a curved pMUT of the present disclosure. The process flow starts with FIG. 10a, which shows silicon wet etching using hydrofluoric, nitric, acetic acid (HNA) to form the curved device structural base (e.g., a curved depression in the substrate surface). The etched silicon substrate 30 has a cavity. A low stress nitride 31, by example a 1.2 μm thick low stress, low-pressure chemical vapor deposited (LPCVD) nitride, is used as a hard mask for making the etched cavities in silicon using HNA wet etching, which can be an isotropic process.

After making the cavities, as shown in FIG. 10a, the top low-stress nitride 31 is removed. Oxide layer 6 is then deposited. Oxide layer 6 may be a low temperature oxide, typically SiO₂. This produces the bottom layer, e.g., the support layer of the curved pMUT. For example, low temperature oxide (LTO) may be deposited on top and bottom of the wafer. In some cases, this can be a 1.1 μm-thick LPCVD LTO, which is grown to form the backside etching stop layer.

As described herein, oxide layer 6 is then overlaid by the first molybdenum layer 18, followed by aluminum nitride layer 22, then the second molybdenum layer 20. For example, the molybdenum-aluminum nitride-molybdenum stack may be sputter deposited. In one example, the sputtering of the active stack of molybdenum-aluminum nitride-molybdenum can be at thicknesses of 100 nm, 2 μm and 100 nm, respectively. The molybdenum layers 18 and 20 form the bottom and top electrodes, respectively, while the aluminum nitride layer 22 is the piezoelectric and main structural layer of the curved pMUT.

After completing the fabrication of the intermediate structure shown in FIG. 10b, etching is performed as shown in FIG. 10c. Second molybdenum layer 20 and aluminum nitride layer 22 are etched away, providing a via for access to first molybdenum layer 18. FIG. 10c shows the via opening to the bottom electrode, which can be made by SF₆ plasma etching of the top Mo electrode, followed by a combination of plasma dry etching in chlorine-based gases and MF-319 developer wet etching of the AlN layer.

As shown in FIG. 10d, membrane 1 is released using backside deep reactive ion etching to produce cavity 8 with clamp parts 12. The electrical connection is shown diagrammatically. As shown in FIG. 10d, the deep reactive ion etching (DRIE) for backside deep RIE is used to produce cavity 8 and release membrane 1 from the backside. The average diameter of the released membrane is defined by the backside etch opening process.

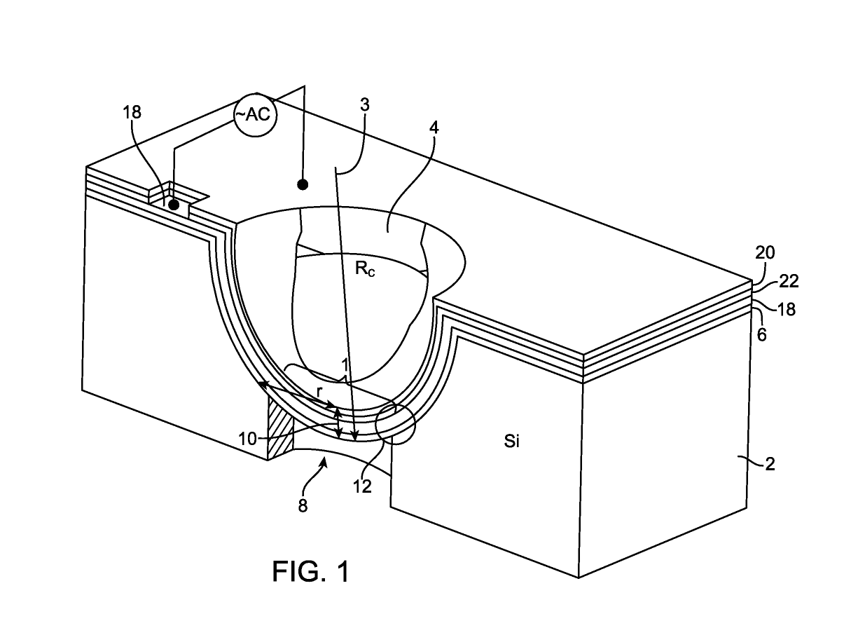

In certain embodiments, the method of making the curved pMUT is CMOS-compatible. For example, aluminum nitride is a CMOS-compatible material. Additionally, the diameter and the radius of the curvature can be controlled through the fabrication process (e.g., the etching steps as described above). The diameter of the curvature, or the average diameter, and the radius of curvature are shown as R_{C} in FIG. 1. By defining the opening size of the HNA wet etching on a low-stress nitride, and by controlling the time, the radius of the curvature can be controlled. By defining the diameter of the backside hole, the size of the average diameter of the membrane can be defined. This allows fabrication of a device with a defined resonant frequency suitable to a particular desired purpose.

The thickness of the piezoelectric stack can be controlled during the fabrication process by timed sputtering deposition of aluminum nitride. More time produces a thicker aluminum nitride. The curvature and diameter of the membrane can be controlled with different fabrication parameters, such as time, and the combination of the HNA etching process. Embodiments of the presently disclosed fabrication system may facilitate control of the curvature and the size of the membrane, which provides the ability to tune the produced curved pMUT to a desired resonant frequency for a particular desired purpose.

Other methods of making a curved piezoelectric transducer may be employed. For example the method of making a curved piezoelectric transducer may include producing the curved piezoelectric transducer through a self-curving process. By “self-curving” is meant that the curved piezoelectric transducer adopts a curved conformation during the fabrication process without forming a curved depression in the substrate or without the external application of a force to the support layer or piezoelectric transducer during fabrication. A self-curving piezoelectric transducer may spontaneously adopt a curved conformation during the fabrication process. For example, as described herein, a support layer of the piezoelectric transducer may include a central portion and a peripheral portion, where the central portion and the peripheral portion are composed of different materials. In certain instances, the central portion of the support layer may have residual tensile stress. For instance, the central portion of the support layer may be composed of a material having residual tensile stress. Tensile stress (or tension) is stress that leads to expansion. Thus, a central portion of the support layer that has residual tensile stress tends to exert an outward expansion force. In some instances, the peripheral portion of the support layer may surround the periphery of the central portion as described herein. The peripheral portion of the support layer may have residual compressive stress. For example, the peripheral portion of the support layer may be composed of a material having residual compressive stress. Compressive stress is stress that leads to a smaller volume. Thus, a peripheral portion of the support layer that has residual compressive stress tends to exert an inward compression force.

In some embodiments, the central portion and the peripheral portion of the support layer are deposited on the surface of the substrate where the substrate has a substantially planar surface (e.g., not in a curved depression). As described above, the central portion of the support layer may have residual tensile stress and the peripheral portion of the support layer may have residual compressive stress. In these embodiments, the method includes removing substrate material from an opposing surface of the substrate. The opposing surface may be a surface opposite from the substrate surface where the support layer is deposited. In some instances, removing substrate material from the opposing surface of the substrate produces an opening (also referred to as a via or a hole herein) through the substrate. As described herein, the opening through the substrate may expose a portion of the support layer. For instance, the exposed surface of the support layer may be the surface of the support layer opposite from the surface where the piezoelectric element will be deposited. In certain instances, the interaction of the residual tensile stress of the central portion and the residual compressive stress of the peripheral portion causes the support layer to adopt a curved conformation, thus producing a curved support layer.

In certain embodiments, after formation of the curved support layer, the method of making the curved piezoelectric transducer further includes depositing a curved piezoelectric element on the curved support layer. As described herein, a piezoelectric element may include multiple layers, such as a first electrode layer, a piezoelectric layer, and a second electrode layer. The layers of the piezoelectric element may be deposited on the curved support layer to form a curved piezoelectric element on a surface of the curved support layer.

As described above, after formation of the curved piezoelectric element on the curved support layer, the method of making the curved piezoelectric transducer further includes forming an electrical contact to the first electrode layer of the piezoelectric element, and forming an electrical contact to the second electrode layer of the piezoelectric element. To form an electrical contact to the first electrode layer, the method may also include removing a portion of the overlying piezoelectric layer and the overlying second electrode layer to form a hole or via exposing a portion of the surface of the first electrode layer. In some instances, removing a portion of the overlying piezoelectric layer and the overlying second electrode layer includes etching hole or via to expose a portion of the surface of the first electrode layer.

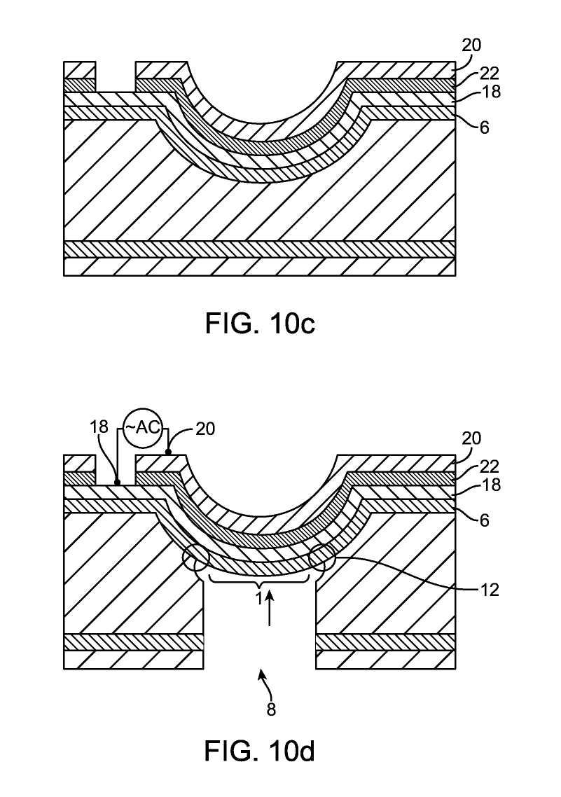

Other methods of making a curved piezoelectric transducer are also possible. For example, a method of making an array of curved piezoelectric transducers is shown in FIGS. 12a-12f, which illustrate a flow diagram of a thermal deformation fabrication technique for an array of curved pMUT devices. The curved pMUT device shown singly in FIG. 10 is provided in multiples in FIG. 12 as an array of curved pMUT devices.

As shown in FIG. 12a, an aspect of this fabrication approach is to engineer the curvature of the membrane. One technique for making this array is to start with a silicon on insulator (SOI) wafer 46, which has an oxide layer 40 above which is a support layer 44, and below which is handle layer 42. Thus, oxide layer 40 is between support layer 44, and handle layer 42.

As shown in FIG. 12b, backside etching is then performed, which naturally stops on the oxide 40, as described in the single device fabrication description, above. Handle layer 42, oxide layer 40, and support layer 44 of the original SOI wafer 46, are retained. The backside etching step produces etched empty areas 50 which serve to define the membranes 48.

As shown in FIG. 12c, the oxide layer 40 is removed from etched empty areas 50 in an optional step. Then, ΔP 52, a pressure, is applied.

FIG. 12d shows the fabrication where ΔP 52, a pressure, is applied, causing membranes 48 to deflect in response to mold 57. This deflection is facilitated by heating the modified SOI wafer 46 to an increased temperature 56.

The SOI wafer 46 is then slowly cooled to room temperature. At that stage, a deformation is formed in the support layer 44, and mold 57 is removed. The piezoelectric stack is then deposited as shown in FIG. 12e where a thin layer of oxide 58, is deposited. The thin layer of oxide 58 can be used later as a stop layer.

The result of this processing is shown in FIG. 12f. The silicon can be etched using backside etching. For example, the method may include depositing a curved pMUT using molybdenum-aluminum-molybdenum on a curved silicon support layer 44. The curved support layer may be composed of silicon, or can be any membrane that can be curved, such as an oxide or metal.

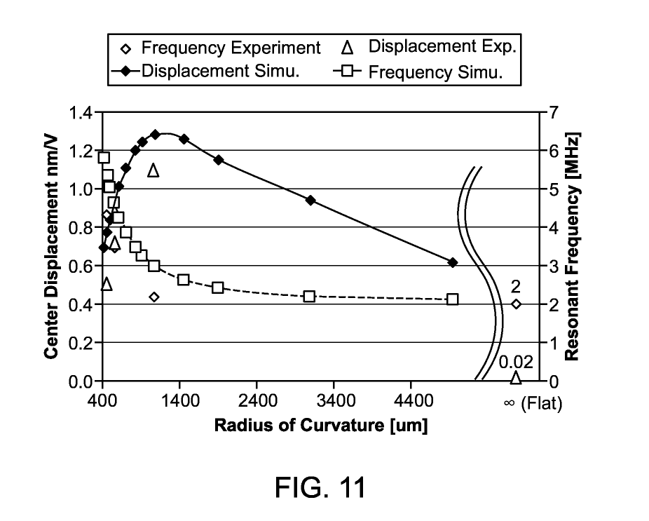

FIGS. 13a-13d provide a flow diagram of an additional fabrication approach. FIG. 13a shows silicon wafer 46, which serves as the substrate for the curved pMUT. FIG. 13b shows a shallow etch into the silicon wafer. Oxide 57 is then deposited. This structure is then bonded to another wafer, which, in some embodiments, may be thinned down to produce a membrane 60 of a certain desired thickness, t, and a certain desired diameter, d.

As shown in FIG. 13c, voltage, V, is applied between the top and bottom. As a result, the membrane 60 will deflect, producing curvature. While continuing to apply voltage, a top material 62, such as an oxide, is deposited.

In FIG. 13d, the next step is the deposition of first molybdenum layer 18, followed by aluminum nitride layer 22, then second molybdenum layer 20. At this stage the bottom silicon layer can be removed. The voltage can be varied to control the curvature. Additionally, pressure can be varied to control the curvature.

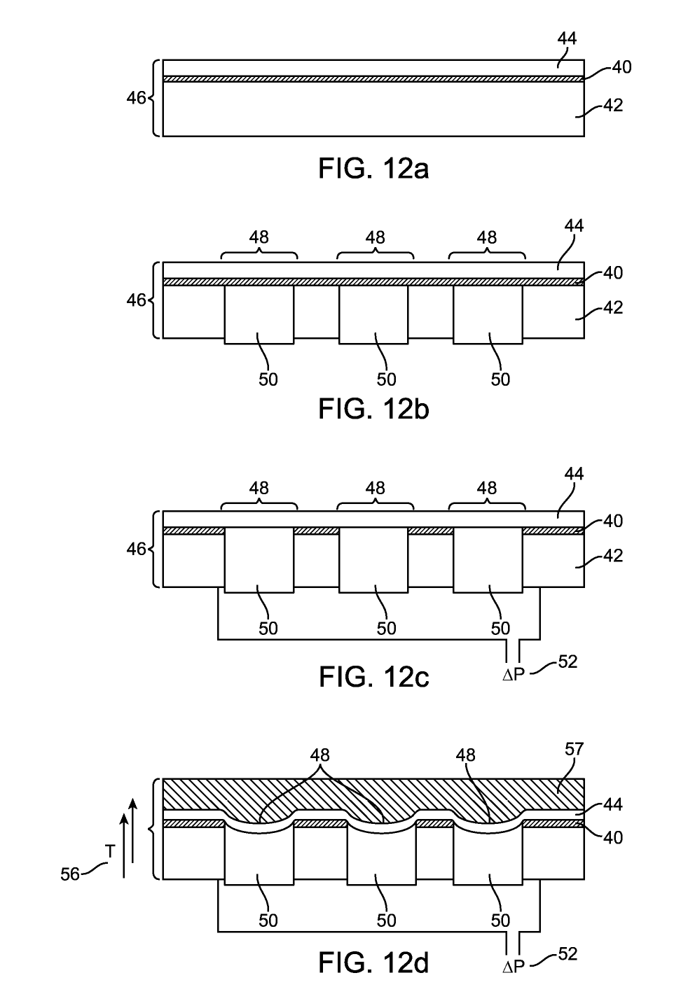

FIG. 14 shows an alternate fabrication approach using a mandrel. A thin oxide membrane 63 is pressed down upon by silicon mandrel 65. Pressure is applied, and thin oxide membrane 63 is deformed. This produces a curved support layer upon which a piezoelectric element may be deposited as described above. If the position where the pressure is applied is varied, specific curvatures can be produced. While the surface is deformed, heat may be applied to set that shape.

### Silicon Bending Fabrication

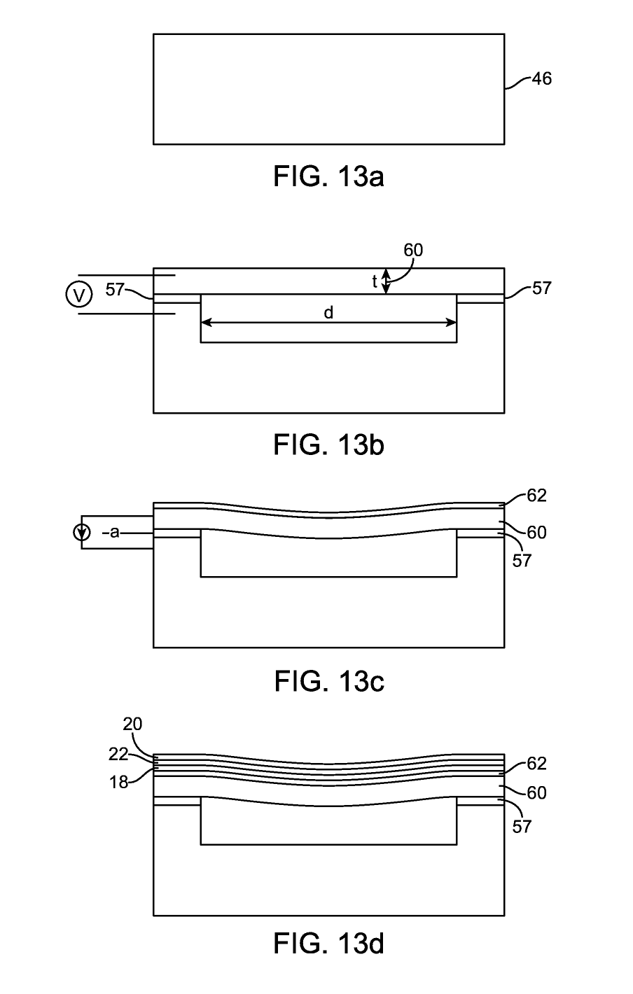

FIGS. 16a-16e illustrate an example of an additional fabrication process for the curved pMUT which is different from the wet etching for silicon described above in FIG. 10. In this case, silicon bending is employed to fabricate curves. As shown in FIG. 16a, a layer of low stress silicon nitride (LSN) 67 is deposition on top of silicon substrate 2. A layer of low temperature oxide 68 is deposited over silicon nitride 67. The layer of oxide 68 is then subjected patterning 69, which uncovers a surface on silicon nitride 67 to either side of patterned layer of oxide 68. A thin sacrificial layer of phosphosilicate glass (PSG) is then deposited and patterned 77. Structural layer 70 is then deposited, which can be composed of such materials as polysilicon or silicon nitride.

In this fabrication process, the layer of oxide 68 serves as a step to increase the height of the structural layer 70. As seen below, this allows the structure to be bent at a later stage, releasing the structure.

As shown in FIG. 16b, an opening 71 is then patterned through structural layer 70, providing access to the oxide layer 68 beneath the structural layer 70. The resulting via produced by opening 71 provides access to the oxide layer 68. Cavity 72 is filled with oxide 68, which will be etched away where it is patterned above the substrate surface.

As shown in FIG. 16c, the oxide layer 68 beneath the structural layer 70 may be etched away, such as by hydrogen fluoride (HF) vapor or other means. Plasma enhanced chemical vapor deposition (PECVD) oxide is used simultaneously to make the vacuum in the cavity 72, and also block any possible holes. The result is the blockage of opening 71. A vacuum is provided beneath the structural layer 70. The force of the vacuum causes the structural layer 70 to bend. This bent region of structural layer 70 serves as the support layer of the curved pMUT.

As shown in FIG. 16d, sequential layers of first molybdenum layer 73, aluminum nitride layer 74, and second molybdenum layer 75 are sputtered on the structural layer 70. These layers conform to the curved area of structural layer 70, which is curved due to the vacuum beneath it.

As shown in FIG. 16e patterning may be performed to produce an access opening 76 to the bottom electrode and isolate the top electrode in cases where appropriate. The structure produced according to FIG. 16 is curved. In some instances, the result is a structure that acts as a tube for the wave, that is, as a wave confiner.

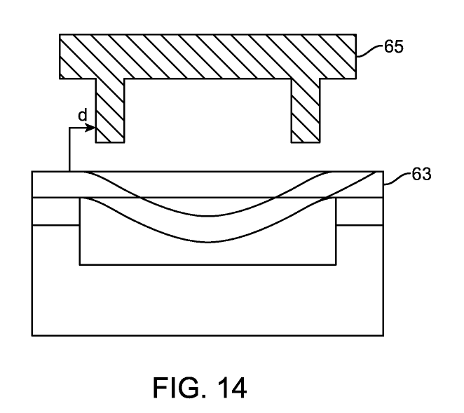

As shown in FIG. 17, the top schematic shows the result of the first fabrication method described above in FIG. 10. In some instances, the result is upper structure 77 of almost a constant radius. However, as shown in the lower structure 78 of FIG. 17, the fabrication method of FIG. 16 may produce a curved pMUT which does not have a constant radius, but rather has double curvatures, with two radiuses. In certain embodiments, the double curvatures structure has an greater response than the curved pMUT structure having substantially constant radius.

In contrast to the fabrication method of FIG. 10, in the fabrication method of FIG. 16, instead of having a backside cavity to reach the diaphragm, there is a structural layer which has a hole, or cavity, beneath it with the curved pMUT suspended over the substrate. When the oxide layer is removed and the structure is subject to a vacuum so that it bends, it is released from the backside.

This backside etching results in the formation of a tube, which acts as a wave-confiner. In function, the curved pMUT emitted wave is confined inside a tube, rather than propagating in all directions. As a result, by example as in the case of a pMUT fingerprint ID system, nearly all the acoustic waves confined in the tube propagate to the user's finger directly, regardless of how large the beam width for the original curved pMUT. In some instances, this facilitates an increase in the directionality of the sensor.

This directionality also serves to focus the energy, providing greater beam penetration and further distance, increasing the range of the device with the creation of a focal point. Without directionality, the curved pMUT wave may propagate in many directions. However, with directionality, if there is a focal point, the energy can be concentrated towards the focal point. Thus, this design can facilitate an increase in the output acoustic pressure, which in turn may produce a higher response. The fabrication shown in FIG. 16 may also be performed without the use of HNA wet etching.

Aspects of the present disclosure include methods of using the curved piezoelectric transducers disclosed herein. In some embodiments, methods of using a curved piezoelectric transducer include producing sound waves from a curved piezoelectric transducer, where the curved piezoelectric transducer is configured to direct the produced sound waves to a target. In some cases, the produced sound waves are ultrasound waves. The target for the produced sound waves may be any desired target and may depend on the type of device being used. For instance, as described herein devices that include a curved piezoelectric transducer may include sensor devices, and as such the target may be the subject being sensed by the sensor device, such as, but not limited to, a finger (e.g., a fingerprint), a liquid, an automobile, a person, an animal, or any other target that may be detected by the sensor device. Other devices that may include a curved piezoelectric transducer are ultrasonic transducer devices, and as such, the target may be a substrate or liquid being treated or modified by the ultrasound waves produced by the ultrasonic transducer device.

### Examples of Additional Curved Piezoelectric Transducer Parameters

The examples provided below use molybdenum and aluminum nitride as the materials for the electrodes and piezoelectric layer of the piezoelectric element, respectively. While many other materials can be used in the fabrication of the curved pMUT, parameters of these particular materials are representative of the curved pMUTs of the present disclosure.

In some of the embodiments of the curved pMUT, the thickness of the electrodes (e.g., molybdenum layer thickness) range from 10 nm to 500 nm, such as 20 nm to 300 nm, or 50 nm to 200 nm, for instance 100 nm. The piezoelectric layer (e.g., aluminum nitride layer) in these curved pMUT embodiments may be 0.5 μm to 5 μm, such as 1 μm to 4 μm, for example 2 μm.

Resonant frequencies achieved by particular embodiments of the curved pMUT range from 0.1 MHz to 100 MHz, such as 0.1 MHz to 80 MHz, or 0.5 MHz to 50 MHz, or or 0.5 MHz to 40 MHz, or 1 MHz to 3 MHz, such as for example 2 MHz.

FIG. 1 shows a drawing of a three dimensional cross-section providing an example of a curved pMUT of the present disclosure. Although the curved structure is drawn with downward deflection (concave) at the center of the membrane, the same principles and analyses apply to curved structures with upward deflection (convex) at the center of the membrane. As a base for the construction of the curved pMUT membrane 1, a silicon wafer 2 is provided. A silicon nitride mask (not shown) is used to provide certain features during manufacture. The line designated “R_{C}” is the radius of curvature 3 of the curved pMUT structure. The physical feature of curvature 4 is engineered into silicon wafer 2 by removal of material from the silicon wafer, such as through a hydrofluoric, nitric, acetic acid (HNA) etching process. The average diameter is defined through back-to-front side alignment through a silicon etch process. This process is stopped on oxide layer 6, which is typically SiO₂. The radius of curvature 4 is provided via HNA wet etching. The average diameter is defined via STS deep reactive ion etching from the back (bottom) surface of the silicon wafer. This process provides front-to-backside alignment, terminating at the oxide layer 6, and extending through the thickness of the silicon wafer.

At this stage, the curved pMUT structure is formed via a 3-stack layering of a first electrode layer, a piezoelectric layer, and a second electrode layer. The 3-stack layers are composed of molybdenum, aluminum nitride, and molybdenum, respectively. As shown in FIG. 1, the final thickness of the completed pMUT membrane 1 includes the oxide layer 6, overlaid by the first molybdenum layer 18, followed by aluminum nitride layer 22, then the second molybdenum layer 20.

Clamp parts 12 are provided at the edge of curved pMUT membrane 1 at the boundary condition, which is thus clamped. Membrane 1 is the part of the sphere between the clamped circle produced by clamp parts 12 and the backside hole 8. The backside hole 8 is fabricated with deep reactive ion etching to release the membrane as described above.

The curved pMUT has an engineered curvature by defining R_{C}, which is the radius of curvature 3, and defining the average diameter of backside hole 8. The average stack thickness is shown in FIG. 1 as double headed arrow 10. The average stack thickness 10 is about 0.1 μm to 20 μm, such as 1 μm to 4 μm, for example 2 μm.

When functioning, the curved pMUT has an AC voltage applied between the bottom of pMUT membrane 1 at the first molybdenum layer 18 and top of pMUT membrane 1 at the second molybdenum layer 20. The applied voltage causes the membrane to move. When the resonance frequency is reached, the largest deflection is achieved, and the membrane starts to resonate and oscillate.

The curved pMUT can be constructed based on a CMOS compatible process. A concave diaphragm, with a radius of curvature of R_{c} is one embodiment fabricated by etching a cavity into the silicon substrate as described above. The average diaphragm size is determined by the backside through-hole etching process, with an opening radius, r, and the rest of the curved surface serves as an acoustic reflector/concentrator which can further enhance the transduction performance.

As provided herein,
r=average radius
d=average diameter
d=2r

The curved pMUT radius of curvature 3 can range from 50 μm to 8000 μm, such as from 400 μm to 2000 μm. The average diameter can range from 10 μm to 1 mm, such as from 120 μm to 180 μm.

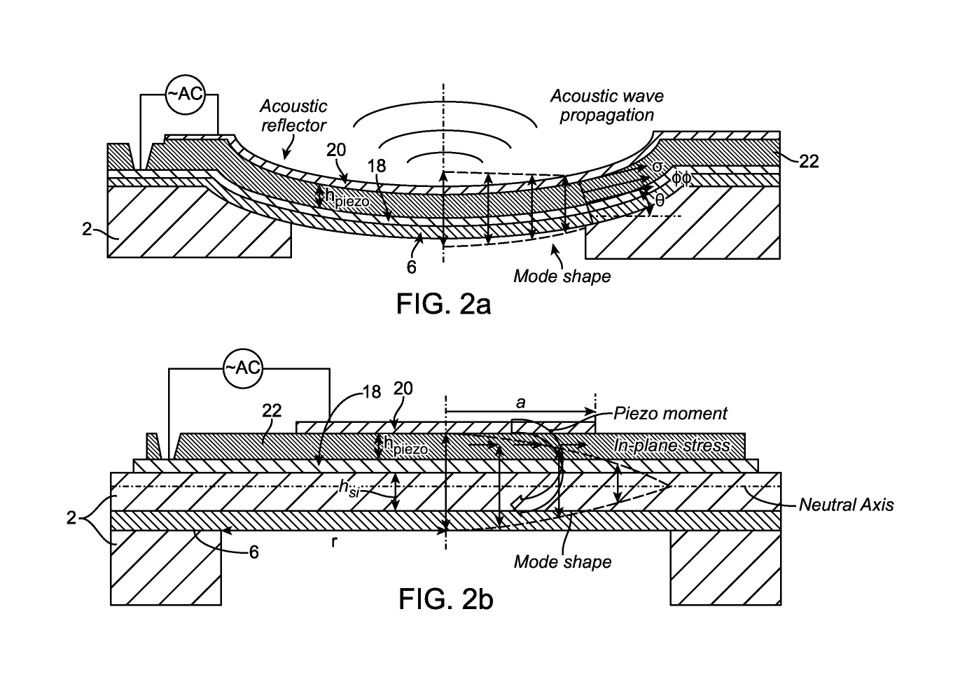

FIG. 2a shows a schematic diagram of a curved pMUT according to the present disclosure. This schematic cross-sectional view of a curved pMUT shows how the curved conformation of the curved pMUT promotes the conversion of in-plane stress ‘σ_{φφ}’ to vertical mechanical forcing function. The curved conformation of the curved pMUT of the present disclosure is contrasted to FIG. 2b, which shows a typical planar pMUT structure.

The generated piezoelectric moment is expressed as:

M^{P}=Y′₀d′₃₁ZV

where Y′₀, is the modified Young's modulus, d′₃₁ is the modified piezoelectric charge constant, Z is the distance of the piezoelectric layer to the neutral axis, and V is the applied voltage. The piezoelectric layer in this example is an aluminum nitride layer 22. In order to excite a planar pMUT, the Laplacian of the piezoelectric moment about the neutral axis of the structure should be non-zero. As a result, an additional structural layer, e.g., silicon is shown here as an example, is needed to generate a non-zero piezoelectric moment for the planar pMUT.

In contrast to a planar pMUT which relies on the excessive plane strain due to the d₃₁ effect to induce vertical deformation, the induced piezoelectric in-plane strain has a vertical component, which is in the direction of the normal motion, as illustrated in FIG. 2a. Hence, the curved-shape diaphragm promotes the conversion of in-plane strain to vertical mechanical motion for higher electromechanical coupling and acoustic pressure. A simplified formula is derived for the uniform normal pressure generated on a piezoelectric hemispherical shell under applied voltage V:

p_{piezo}=2Y′₀d′₃₁V/R_{c},

where R_{c} is the radius of curvature of the diaphragm. The normal driving force helps to eliminate the necessity of the additional structural layer to generate a moment about the neutral axis. As such, the piezoelectric layer alone, in this case aluminum nitride layer 22, can also serve as the structural layer. It is noted that this term goes to zero as the radius of curvature goes to infinity for a planar pMUT.

FIG. 2c is a cross-section view of FIG. 1. The curved pMUT membrane 1 is initially supported in the beginning stages of fabrication on silicon wafer 2. In a sequential layering, the curved pMUT membrane 1 is fabricated from the oxide layer 6, overlaid by the first molybdenum layer 18, followed by aluminum nitride layer 22, then the second molybdenum layer 20. Clamp parts 12 are provided at the edge of curved pMUT membrane 1 at the boundary condition, which is thus clamped. Membrane 1 is the part of the sphere between the clamped circle produced by clamp parts 12 and the backside hole 8. The backside hole 8 is fabricated with deep reactive ion etching to release the membrane as described above.

During use, the curved pMUT has an AC voltage applied between the bottom of pMUT membrane 1 at the first molybdenum layer 18 and the top of pMUT membrane 1 at the second molybdenum layer 20. Aluminum nitride layer 22 serves both as the structural layer and a piezoelectric layer. Aluminum nitride layer 22 provides the main structure for the membrane. Because of its piezoelectric capability, aluminum nitride layer 22 responds to the applied AC voltage.

As shown in FIG. 2c, during fabrication, the curved pMUT does not require patterning of the top electrode (or bottom electrode). By contrast, a typical planar pMUT requires a patterned top electrode. The curved pMUT may be fabricated using a procedure that includes deposition of a molybdenum bottom electrode, an aluminum nitride piezoelectric layer, and a molybdenum top electrode. This fabrication process allows an open contact to the bottom electrode. In the construction of an array of curved pMUT structures, two additional patterning steps may be used to define the top and bottom electrode contacts to form separate electrical contacts to each individual pMUT structure.

FIG. 2d provides a more detailed three-dimensional view of clamp parts 12. In certain embodiments, clamp parts do not rotate or move. Clamp parts 12 may be formed by contact of the curved piezoelectric transducer with the underlying substrate. Because the curved piezoelectric transducer contacts the underlying substrate at the clamp part region, the curved piezoelectric transducer at the clamp part region may not significantly rotate or move during operation of the curved piezoelectric transducer. Clamp parts 12 may be provided in various structures depending on the design of the curved pMUT. For instance, the clamp parts 12 may be provided as a circle, a cylinder, or a double-curved cylinder.

FIG. 2e shows variations on the clamped section. By example, the clamped section could be a cylinder in shape, producing single curvature 14, with clamped boundary condition. Another variant is a double edge, resulting in a double curvature 16. Because these structures are curved, they will produce similar effects and provide a stop against which the curved piezoelectric transducer does not significantly rotate or move as described above. In some cases, the curved pMUTs may be configured as an elongated element if a 1D array is desired.

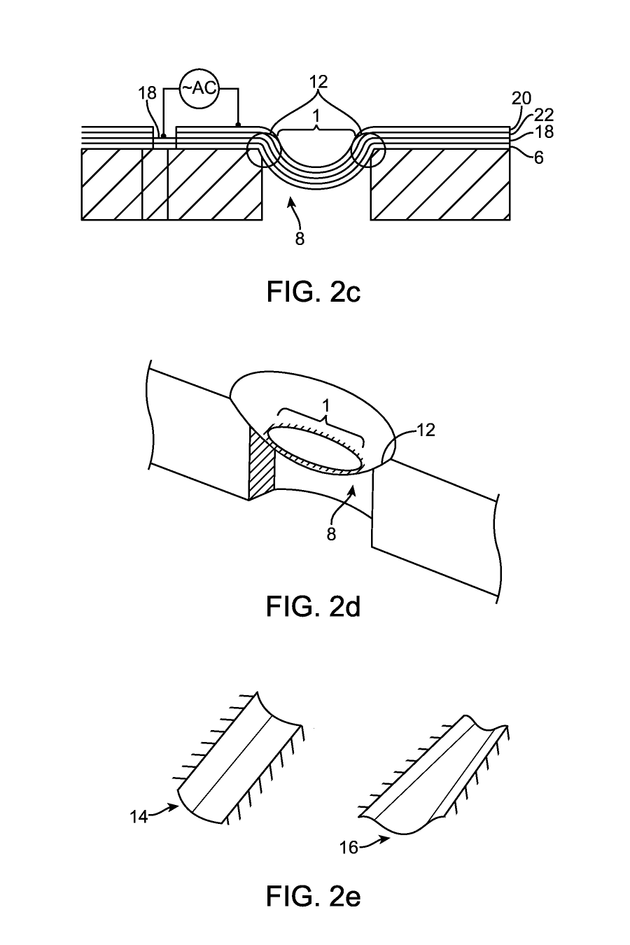

FIG. 3a provides a cross-sectional view of the motion of membrane 1. Clamp parts 12 are provided at the edge of curved pMUT membrane 1 at the boundary condition, which is thus clamped. As shown in FIG. 3b, during use, the curved pMUT may have an AC voltage applied between the bottom of pMUT membrane 1 at the first molybdenum layer 18 and the top of pMUT membrane 1 at the second molybdenum layer 20. As described above, the second molybdenum layer 20 serves as the top electrode for the curved pMUT and the first molybdenum layer 18 serves as the bottom electrode for the curved pMUT.

If an AC voltage is applied, movement is induced in membrane 1. For example, membrane 1 may move up and down around its static point. If a DC voltage is applied, membrane 1 will be deflected. The whole membrane 1 from beneath the clamp points will vibrate under the right input driving voltage and frequency. In certain embodiments, the polarization of the piezoelectric material is perpendicular to the curvature of the curved pMUT. The direction of the motion of membrane 1 may generally be up and down, but may also include complex motions, such as, but not limited to a squiggle-type motion. In some cases, the crystal orientation in the aluminum nitride layer influence the direction of motion.

FIG. 3b provides a cross-sectional view of the motion of membrane 1, in this case showing a second form factor. In this view, curved pMUT membrane 1 is shown as being fabricated from a support layer (e.g., an oxide layer (not shown)), overlaid by the first molybdenum layer 18, followed by aluminum nitride layer 22, then the second molybdenum layer 20. Clamp parts 12 are provided at the edge of curved pMUT membrane 1 at the boundary condition, which is thus clamped. The support layer (e.g., oxide layer (not shown)) is employed as a stop layer, e.g., a layer that stops the etching process of the backside hole from causing significant etching of the curved piezoelectric transducer.

If a suitable AC voltage (in magnitude and frequency) is applied to the second molybdenum layer 20, serving as the top electrode, and the first molybdenum layer 18, serving as the bottom electrode, the membrane starts to resonate, moving with an up and down motion. As shown in FIG. 3b, the direction of motion is perpendicular to the center. Both the amplitude and displacement may depend on the magnitude of the voltage that is applied, and the frequency of operation. With the application of a DC voltage, the curved piezoelectric transducer may have a DC response of about 1 nm/V. At resonance, the curved piezoelectric transducer may have a DC response in the range of 50 nm/V or more in air.

### Concave and Hybrid Concave-Convex Surface

A concave or hybrid concave-convex structure to the curved pMUT can be engineered during manufacture. The concave or hybrid concave-convex structures can provide capabilities, such as acoustic focus, in some cases where an antenna assists with the focusing of the acoustic energy. The acoustic pressure may be related to the displaced volume. If the membrane moves a certain distance, as long as it is the same volume displacement, the same acoustic pressure may be achieved regardless of the specific membrane shape. In certain instances, the curvature of the curved pMUT facilitates focusing of the acoustic waves produced by the device. For example, the curvature of the curved pMUT can be hemispherical, elliptical, parabolic, etc. In some instances, a parabolic curved pMUT may focus acoustic waves in parallel, rather than to a point, thus facilitating a reduction in crosstalk between individual pMUTs in an array of pMUTs. In some instances, focusing of the acoustic waves provides from a concentration of the acoustic pressure by reducing radiation of the acoustic pressure in all directions. This focusing effect can provide the signal additional power.

In designing arrays of curved pMUTs, a consideration is that if a wave is sent through one element, it is useful to avoid a response from another element. This would constitute crosstalk between the elements. With the concave structure of the curved pMUTs, crosstalk between the elements may be reduced. This provides the opportunity, for instance, to change radiating energy from 180 degrees to 20 degrees, which may facilitate an increase in the directionality of the acoustic waves, for instance by 2× or more, 4× or more, 6× or more, 8× or more, 10× or more, or 20× or more.

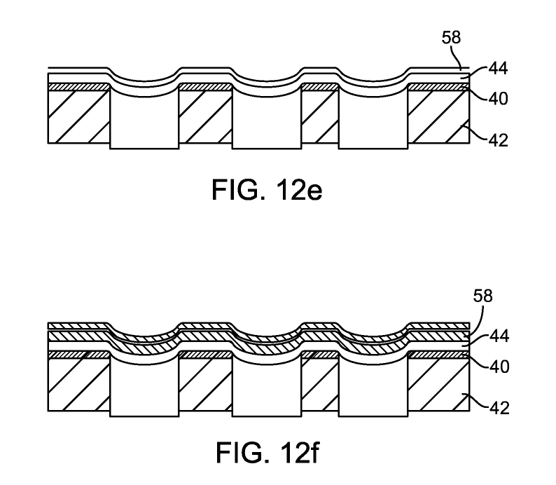

As shown in FIG. 15 when considering the direction of the acoustic wave, in some cases, the initial cavity backside alignment may be centered as shown in backside alignment 102, producing an un-angled acoustic beam 103. However, if the backside alignment is purposefully offset as in backside alignments 104 and 106, then the maximum displacement occurs at an angle, such as angled acoustic beams 105 and 107. The angle of these acoustic beams can be predetermined. This can be accomplished by offsetting the backside to the front-side alignment, providing directionality of the beam An enlargement of alignment 106 with angled acoustic beam 107 is also provided in FIG. 15.

In an array formation as shown in FIG. 15, the resulting angled acoustic beams focus to a single point. This is a designed offset alignment in order to achieve a focusing of the waves at a single point. In some embodiments of the curved pMUT arrays, the angle of the beams can be tuned electronically to change the focus of the point. If there is a desired focus point, a delay can be provided so that the separate curved pMUTs in the arrays will focus at another point. This electronic tuning approach can be used to produce a scanning effect by changing the focus. In some cases, a lens may be used to produce or supplement the scanning effect. For example, a lens that can be moved may be used to produce or supplement the scanning effect.

### Vibration Theory of Elastic PMUT Shells

Aspects of the present disclosure also provide for curved pMUT diaphragms having an increased radial deflection per unit input voltage, as follows:
(1) theoretically-derived differential equations governing forced vibration of a spherical piezoelectric shell polarized in a direction perpendicular to its curvature;
(2) closed-form solutions for the forced vibration equations under both radial pressure and electric potential with clamped boundary conditions; and
(3) explicit predictions of the axisymmetric radial displacement shape function of curved pMUT with respect to the tangential angular position, structural layer thickness, radius of curvature, and average radius of the curved diaphragm.

FIG. 2a illustrates a cross-sectional view of a pMUT in the transmission mode. When an AC voltage of magnitude Vᵣ is applied between the top and bottom electrodes, an in-plane tension is developed in the piezoelectric layer that serves both the piezoelectric and the structural functions. The in-plane strain can be converted to vertical mechanical motion with the assistance of the curved diaphragm for high electromechanical coupling. The out-of-plane vibration of the curved pMUT causes the transmission of an acoustic wave of pressure pᵣ.

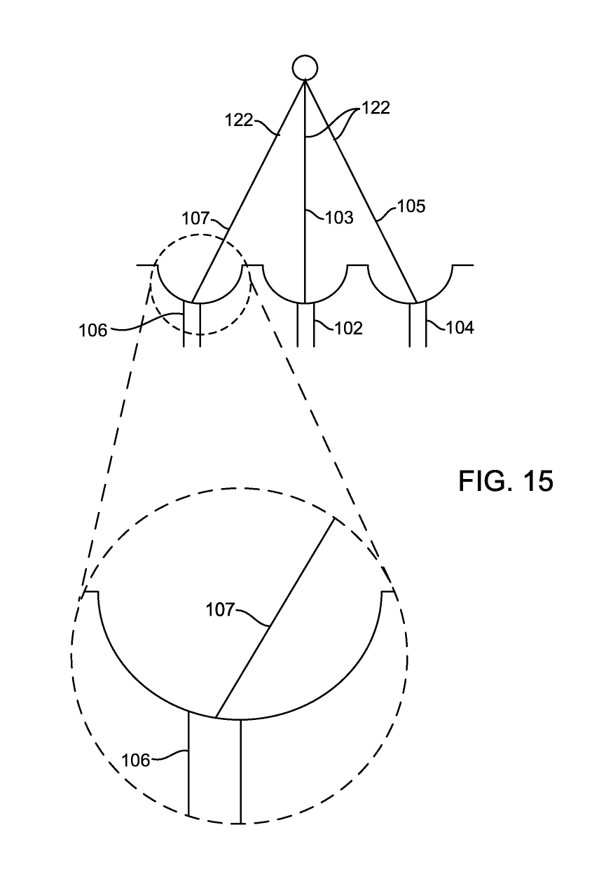

Using the spherical coordinate system (r, θ, φ), a magnified view of the volume element is shown in FIG. 18a along with the stress couples and stress resultants. Since the thickness of the curved pMUT is small compared with the other surface dimensions (h/R_{C}<<1), Love's first approximation theory is appropriate for the geometrical and dynamic analysis of this device.

The transverse normal to the middle surface remains straight and normal to the deformed middle surface such that the transverse shear strains are infinitesimal (ε_{rθ}≈0 & ε_{rφ}≈0) and all nonlinear terms can be neglected. It is also assumed that the transverse normal is inextensible and the transverse normal strain is negligible (εᵣᵣ≈0). The total strain of a spherical shell εij in the i- and j-directions can be decomposed into εij⁰ and flexural strains εij

εᵢⱼ=εᵢⱼ⁰+ζεᵢⱼ¹ where i,j∈{θ,φ} (1)

where ζ is the radial distance from the center of the pMUT diaphragm as shown in FIG. 18b. By applying the Love-Kirchhoff's assumption, strain in spherical coordinates can be expressed as a function of the displacement vector components and their derivative in spherical coordinates (only the φφ component is shown for simplicity)

$${ɛ}_{φ φ}^{0} = \frac{1}{{R}_{c}} ( \frac{\partial {u}_{φ}}{\partial φ} + w ) \qquad ( 2 )$$

where uφ, and w are the displacement vector components in the φ-, and r-directions, respectively. Furthermore, the piezoelectric material is modeled as an isotropic material, where the Young's modulus Y₀ and the Poisson's ratio v are the only two independent variables required to represent the mechanical properties. Using Love's approximation, the transverse shear stresses are negligible (σ_{rθ}≈0 & σ_{rφ}≈0), and the transverse normal stress is small compared to the other normal stresses (σᵣᵣ≈0). Since the external electric field Eᵣ is applied along the polarization direction, the transverse piezoelectric charge constants d_{rθ} and d_{rφ} are assumed to be equal with the magnitude of d₃₁. The constitutive equations for a linear isotropic piezoelectric medium relating the tangential stresses of a curved pMUT with constant radius of curvature can be derived (only the φφ component is shown for simplicity):

$${σ}_{φ φ} = \frac{{Y}_{0}}{1 - {v}^{2}} [ {ɛ}_{φ φ} + v {ɛ}_{θ θ} - ( 1 + v ) {d}_{31} {E}_{z} ] \qquad ( 3 )$$

The stress resultants can be related to the strains and the curved pMUT material and geometric properties by integrating the tangential stresses along the thickness of the curved pMUT and the stress couples can be formulated as well by integrating the infinitesimal stress couple of the respective tangential stress at a distance ζ from the middle surface along the pMUT thickness. The active piezoelectric layer introduces surface forces in the θ- and φ-directions, which are proportional to the applied voltage and piezoelectric charge constant. The motion of the curved pMUT in both transmit and receive modes is rotationally symmetric around the z-axis as shown in FIG. 18b. As such, axisymmetric conditions prevail and ∂/∂θ=0. Using Love's approximation, the dynamic stress equations of a shell with constant radius of curvature and in-plane piezoelectric forces can be expressed as (only the equation in φ is shown for simplicity):

$$\frac{\partial }{\partial φ} ( sin φ {N}_{φ}^{′} ) + \frac{\partial }{\partial θ} ( {N}_{φ θ} ) - cos φ {N}_{θ}^{′} + sin φ {Q}_{φ} = ρ hR sin φ \frac{{\partial }^{2} {u}_{φ}}{\partial {t}^{2}} \qquad ( 4 )$$

where ρ is the density of piezoelectric material; pᵣ is received echo/transmitted acoustic pressure perpendicular to curvature of the diagram; Qθθ and Qφφ, are transverse shear stress resultants; and N′θθ and N′φφ are the modified stress resultant in the θ- and φ-directions, respectively.

$${N}_{φ}^{′} = \frac{{Y}_{0} h}{1 - {v}^{2}} [ {ɛ}_{φ}^{0} + c {ɛ}_{θ}^{0} ] and {N}_{θ}^{′} = \frac{{Y}_{0} h}{1 - {v}^{2}} [ v {ɛ}_{φ}^{0} + {ɛ}_{θ}^{0} ] \qquad ( 5 )$$

The compatibility equation can be derived by the elimination of u_{φ} φ from Eq. (2) and replacing the strains with their resultant stress equivalent using Eq. (5):

$$tan {φ ( {N}_{θ}^{′} - {vN}_{φ}^{′} )}_{, φ} + ( {sec}^{2} φ + v ) {N}_{θ}^{′} - ( v {sec}^{2} φ + 1 ) {N}_{φ}^{′} = \frac{{Y}_{0} h}{R} tan φ [ {w}_{, φ} + w tan φ ] \qquad ( 6 )$$

By going through some mathematical manipulations, the most general form of the governing vibration equation for a unimorph curved pMUT becomes (details are not shown here):

$${∇}^{6} {w}^{*} + {d}_{2} {∇}^{4} {w}^{*} + {d}_{3} {∇}^{2} {w}^{*} + {d}_{4} {w}^{**} = {d}_{5} {Rp}_{ζ} + {d}_{5} \frac{2 {Y}_{0} {d}_{31} {V}_{z}}{( 1 - v )} \qquad ( 7 )$$

where d₂, d₃, d₄, and d₅ are functions of the frequency of operation ω and the stress function F and pMUT material properties and dimensions; w^{*} is the magnitude of the radial displacement w. The free vibration equation can be simplified to product of three Legendre differential equations in spherical coordinates, and the general solution of the radial displacement w_{α} and the particular solution, wₛ, of equation (7) is:

$$\begin{matrix}{w}_{α}^{*} = {A}_{α} {P}_{{1}_{α}} ( cos φ ) + {B}_{α} {Q}_{{1}_{α}} ( cos φ ) α = 1 , 2 , 3 & ( 8 ) \\ {w}_{s}^{*} = \frac{( 1 - v ) + ( 1 - {v}^{2} ) {Ω}^{2}}{[ 2 + ( 1 + 3 v ) {Ω}^{2} + ( {v}^{2} - 1 ) {Ω}^{4} ]} ( \frac{R}{h} ) \frac{1}{{Y}_{0}} [ {Rp}_{ζ} + \frac{2 {Y}_{0} {d}_{31} {V}_{z}}{( 1 - v )} ] & \end{matrix}$$

where P_{lα} and Q_{lα} are the Legendre functions of the first and second kind of order l_{α} respectively. These equations can be solved by boundary conditions. Since the curved pMUT is clamped on its edges, it cannot translate in the r- and φ-directions. In addition, the diaphragm cannot undergo any rotation around φ-axis.

Simulations:

FEM simulations may be used to validate the theoretical model in both displacements per unit voltage and mechanical resonant frequencies. In some embodiments of the simulations, the thicknesses of the metal layers are ignored, and the piezoelectric material is AlN. The mode-shape function, the dynamic response, and the effect of the diaphragm curvature on the displacement amplitude and resonant frequency are extracted from the theoretical model and verified with FEM simulations using COMSOL. In some instances, an example model of a curved pMUT is composed of a 2 μm-thick AlN layer, with an average radius, r, of 70 μm and a radius of curvature, R_{C}, of 1165 μm.

Mode Shapes:

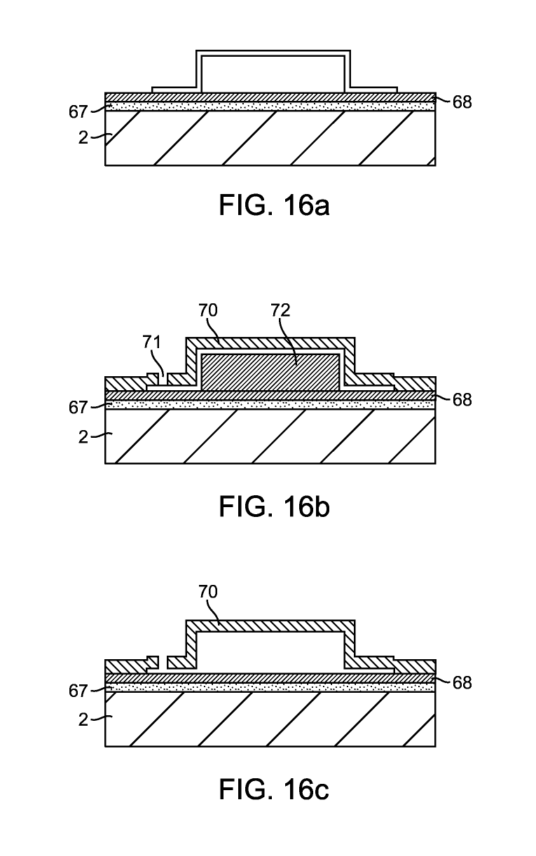

The mode shape function is plotted using the generalized vibration equation as derived in Eq. (8) along with the clamped boundary condition. The radial displacement is normalized with respect to the maximum displacement at the center of the diaphragm as shown in FIG. 19a. The theoretical prediction matches well with the simulated value and the difference is less than 3%, which is due to the fact that AlN is modeled as an isotropic material vs. anisotropic in reality. The mode shape shows an inflection point at an angular position 85% away from the center of the curved pMUT diaphragm versus 60% for its planar counterpart, which is expected to lead to further enhanced volumetric velocity and acoustic pressure emission.

Dynamic Responses:

The frequency response is plotted in FIG. 19b. The analytical model and simulation results on the center diaphragm displacements at low frequencies are calculated as 1.41 nm/V and 1.29 nm/V, respectively, indicating a difference of less than 8.4%. The theoretically calculated resonant frequency is 2.80 MHz, which matches closely to the simulated value of 2.85 MHz. Therefore, the theoretical model and analyses are useful tools to design curved pMUTs to accurately predict their dynamic behaviors.

### Equivalent Circuit Model

It some instances, transducers, especially ultrasonic transducers, may be represented in the form of an equivalent circuit which relates different physics of the device, here electrical, mechanical, and acoustical, to one another. The volumetric displacement and the stored electrical charge of the transducer can be derive in terms of the input voltage and the external pressure explicitly and introduce different elements of the circuit using the derived equations, as discussed below.

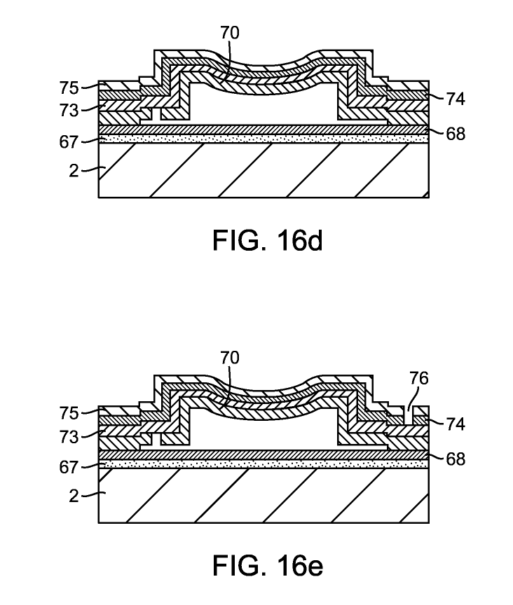

FIG. 20 provides a 2D schematic of an axisymmetric curved pMUT with clamped boundary condition, in a spherical shell of center O and radius R_{c}. The radial and tangential displacements at a point B with an angular position φ from the shell axis are denoted as w(φ) and u_{φ}(φ) respectively. The apex point is denoted as A.

### Volumetric Displacement

The volumetric displacement is the amount of the volume that the transducer under vibration sweeps from its static equilibrium position to its maximum (i.e., final mode shape). For a shell with constant radius of curvature the volumetric displacement can be introduced as the following:

$$\overline{w} = {R}_{c}^{2} {\int }_{0}^{2 π} {\int }_{0}^{{φ}_{0}} w ( φ ) sin φ d φ d θ \qquad ( 9 )$$

where w(φ) is the radial displacement of each point on the middle surface of the transducer. Integration of the radial displacement over the surface area provides the volumetric displacement. Integrating the particular solution in (9) will produce:

$${\overline{w}}_{s}^{*} = 2 π {R}_{c}^{2} [ 1 - cos ( {φ}_{0} ) ] [ {p}_{r} + \frac{2 {Y}_{0} {d}_{31} {V}_{r}}{{R}_{c} ( 1 - v )} ] b ( ω ) \qquad ( 10 )$$

where b(ω) is defined as the following:

$$b ( ω ) = \frac{( 1 - v ) + ( 1 - {v}^{2} ) {Ω}^{2}}{[ 2 + ( 1 + 3 v ) {Ω}^{2} + ( {v}^{2} - 1 ) {Ω}^{4} ]} ( \frac{{R}_{c}^{2}}{{hY}_{0}} ) \qquad ( 11 )$$

Applying the boundary conditions will give the following:

$$( [ \begin{matrix}{P}_{{l}_{1}} ( cos {φ}_{0} ) & {P}_{{l}_{2}} ( cos {φ}_{0} ) & {P}_{{l}_{3}} ( cos {φ}_{0} ) \\ f ( {l}_{1} ) & f ( {l}_{2} ) & f ( {l}_{3} ) \\ g ( {λ}_{1} ) f ( {l}_{1} ) & g ( {λ}_{2} ) f ( {l}_{2} ) & g ( {λ}_{3} ) f ( {l}_{3} )\end{matrix} ] = [ {a}_{ij} ] ) [ \begin{matrix}{A}_{1} \\ {A}_{2} \\ {A}_{3}\end{matrix} ] = [ \begin{matrix}- {w}_{s}^{*} \\ 0 \\ 0\end{matrix} ] \qquad ( 12 )$$

Where the functions f and g are defined as the following:

$$\{ \begin{matrix}f ( x ) = ( x + 1 ) [ csc {φ}_{0} {P}_{x + 1} ( cos {φ}_{0} ) - cot {φ}_{0} {P}_{x} ( cos {φ}_{0} ) ] \\ g ( x ) = [ ( x - 2 ) / 12 ( 1 + v ) {( h / {R}_{c} )}^{2} + 1 ] / [ - x + ( 1 - v ) + ( 1 - {v}^{2} ) {Ω}^{2} ]\end{matrix} \qquad ( 13 )$$

Thus, A_{α}s and the general displacement can be derived from (12):

A_{α}=A′_{α}w^{*}ₛ,w^{*}_{α}=A′_{α}P_{lα}(cos φ)w^{*}ₛα=1,2,3 (14)

where A′_{α}s and the other relevant parameters derived from (12) and (13) are defined and listed in Table 1.
A_{α}s are functions of frequency, material and geometric properties and are proportional to the specific displacement. The total displacement can be derived as the following:

$${w}^{*} = [ 1 + {A}_{1}^{′} {P}_{{l}_{1}} ( cos φ ) + {A}_{2}^{′} {P}_{{l}_{2}} ( cos φ ) + {A}_{3}^{′} {P}_{{l}_{3}} ( cos φ ) ] [ {p}_{r} + \frac{2 {Y}_{0} {d}_{31} {V}_{r}}{{R}_{c} ( 1 - v )} ] b ( ω ) \qquad ( 15 )$$

TABLE 1

| Parameter | Expression |
|---|---|
| A₁′ | −m₁/(a₁₁m₁ − a₁₂m₂ + a₁₃m₃) |
| A₂′ | m₂/(a₁₁m₁ − a₁₂m₂ + a₁₃m₃) |
| A₃′ | −m₃/(a₁₁m₁ − a₁₂m₂ + a₁₃m₃) |
| m₁ | (a₂₂a₃₃ − a₃₂a₂₃) |
| m₂ | (a₂₁a₃₃ − a₃₁a₂₃) |
| m₃ | (a₂₁a₃₂ − a₃₁a₂₂) |

By Integrating (15) over the surface area of the shell on spherical coordinate system using (9), the total volumetric displacement is obtained:

$${\overline{w}}^{*} = 2 π {R}_{c}^{2} [ ( 1 - cos {φ}_{0} ) + {A}_{1}^{′} H ( {l}_{1} ) + {A}_{2}^{′} H ( {l}_{2} ) + {A}_{3}^{′} H ( {l}_{3} ) ] [ {p}_{r} + \frac{2 {Y}_{0} {d}_{31} {V}_{r}}{{R}_{c} ( 1 - v )} ] b ( ω ) {e}^{j ω t} \qquad ( 16 )$$

where H is a function of the Legendre function degrees of (3-48):

$$H ( {l}_{α} ) = \frac{{P}_{{l}_{α} - 1} ( cos {φ}_{0} ) - {P}_{{l}_{α} + 1} ( cos {φ}_{0} )}{1 + 2 {l}_{α}} \qquad ( 17 )$$

### Electric Displacement Field and the Total Charge

The electric displacement field for a clamped, part-of-a-sphere, piezoelectric shell can be written as the following form:

Dᵣ=d₃₁(σ_{φφ}+σ_{θθ})+εᵣEᵣ (18)

Substituting for the strains in (18) provides:

Dᵣ=Y′₀d′₃₁(ε_{φφ}+ε_{θθ})+εᵣ(1−k²)Eᵣ (19)

where Y′₀=Y₀/(1−ν²), d′₃₁=d₃₁(1+ν), and k²=2Y′₀(d′₃₁)²/[(1+ν)εᵣ]. The electric displacement field can be written in terms of the displacements as the following:

$${D}_{r} = \frac{{Y}_{0}^{{ }^{′}} {d}_{31}^{′}}{{R}_{c}} ( cot φ {u}_{φ} + \frac{\partial {u}_{φ}}{\partial φ} + 2 w ) + {ɛ}_{r} ( 1 - {k}^{2} ) {E}_{r} \qquad ( 20 )$$

To calculate the electric charge on the surface of the transducer, the electric displacement field must be integrated over the surface area,

$$( i . e . , Q = {( {\int }^{•} )}_{A} {D}_{r} dA ) .$$

By doing so and considering the clamped condition at the boundary the electric charge

$$Q = 4 π {Y}_{0}^{′} {d}_{31}^{′} {R}_{c} [ ( 1 - cos {φ}_{0} ) + {A}_{1}^{′} H ( {l}_{1} ) + {A}_{2}^{′} H ( {l}_{2} ) + {A}_{3}^{′} H ( {l}_{3} ) ] [ {p}_{r} + \frac{2 {Y}_{0} {d}_{31} {V}_{r}}{{R}_{c} ( 1 - v )} ] b ( ω ) + \frac{2 π ( 1 - cos {φ}_{0} ) {R}_{c}^{2} {ɛ}_{r} ( 1 - {k}^{2} )}{h} {V}_{r} \qquad ( 21 )$$

### Equivalent Circuit Components

The volumetric displacement and the electric charge now can both be written in terms of the input voltage and the external pressure in the following forms using (16) and (21):

w̅*=Yₘpᵣ+bₘₑVᵣ (22)

Q=bₑₘpᵣ+CₑₘVᵣ+C₀Vᵣ (23)

where Yₘ is the mechanical admittance defined as the volumetric displacement per unit input pressure while the input voltage port is shorted. bₘₑ and bₑₘ are mechanical due to electrical and electrical due to mechanical transduction coefficients and are equal which shows that the system is reciprocal. C₀ is the blocked parasitic capacitance and Cₑₘ is the induced capacitance due to the mechanical motion. All of the mentioned parameters are listed in Table 2.

TABLE 2

| Parameter | Expression |
|---|---|
| Yₘ | 2πR_{c}² [(1 − cosφ₀) + A₁′H(l₁) + A₂′H(l₂) + A₃′H(l₃)]b(ω) |
| bₘₑ = bₑₘ | $[ \frac{4 {πY}_{0} {d}_{31} {R}_{c}}{( 1 - v )} ] [ ( 1 - cos {φ}_{0} ) + {A}_{1}^{′} H ( {l}_{1} ) + {A}_{2}^{′} H ( {l}_{2} ) + {A}_{3}^{′} H ( {l}_{3} ) ] b ( ω )$ |
| Cₘ | $\frac{8 {πY}_{0}^{2} {d}_{31}^{2}}{{( 1 - v )}^{2}} [ ( 1 - cos {φ}_{0} ) + {A}_{1}^{′} H ( {l}_{1} ) + {A}_{2}^{′} H ( {l}_{2} ) + {A}_{3}^{′} H ( {l}_{3} ) ] b ( ω )$ |
| C₀ | 2π(1 − cosφ₀) R_{c}²εᵣ(1 − k²)/h |

### Equivalent Circuit Models

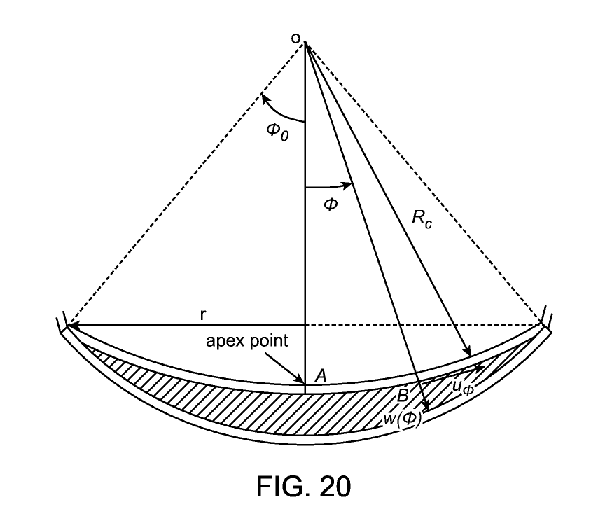

Having the system equations, (22) and (23), and explicit expressions for all the system parameters allows the development of a circuit model for the device showing the correlation between the electrical and mechanical/acoustical domains. Any of the following circuit models shown in FIG. 24 can serve as the equivalent circuit of the transducer. As shown in Equation 24, Zₑ is the electrical feedthrough, Zₘ is the mechanical impedance, Zₐ is the acoustical load, and η is the electromechanical transformer ratio defined as the followings:

$${Z}_{e} = \frac{1}{j ω {C}_{0}} , {Z}_{m} = \frac{1}{j ω {Y}_{m}} , η = \frac{{b}_{t}}{{Y}_{m}} \qquad ( 24 )$$

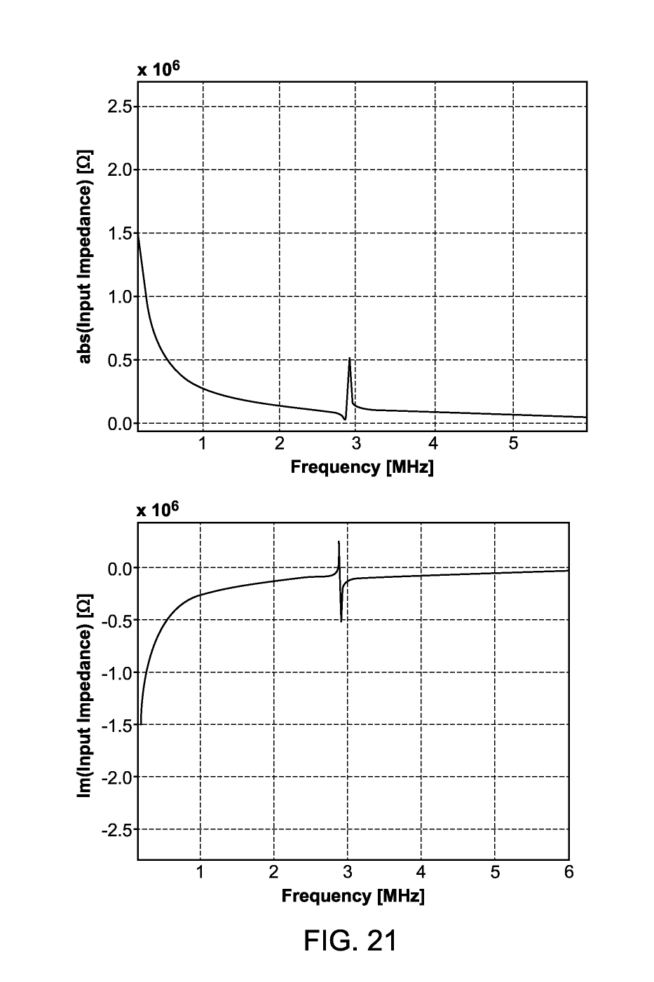

For the case of operation in vacuum, the acoustic impedance can be assumed to be zero, thus the output pressure port becomes shorted (see FIG. 25), and the input impedance would be the parallel combination of the electrical feedthrough and the mechanical impedance transferred to the electrical side

$${Z}_{in} = {Z}_{e} ‖ ( {Z}_{m} / {η}^{2} ) = {Z}_{e} ‖ {Z}_{m}^{′} = \frac{1}{j ω {C}_{0}} ‖ \frac{1}{j ω {η}^{2} {Y}_{m}} \qquad ( 25 )$$

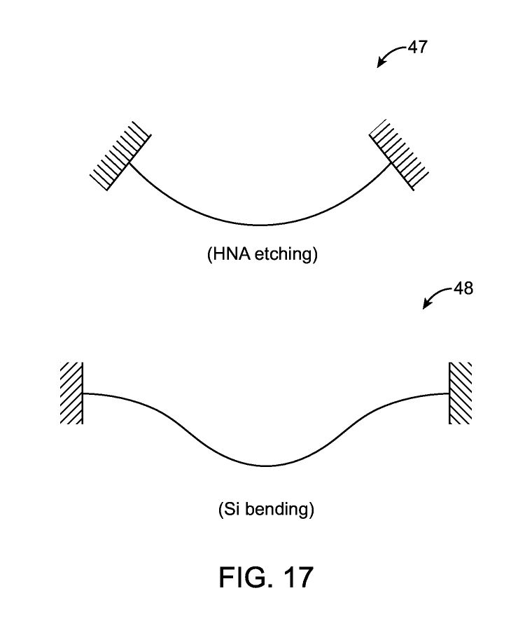

The absolute and imaginary value of the input impedance for a curved AlN pMUT with an average radius of 70 μm and radius of curvature of 1065 μm and AlN thickness of 2 μm operating in vacuum is shown in FIG. 21. FIG. 21 shows graphs of resonant and anti-resonant frequencies of 2.84 MHz and 2.90 MHz that gives an effective electromechanical coupling (K_{eff}²) of 4.3%. Absolute (FIG. 21, top) and imaginary (FIG. 21, bottom) values of the input impedance versus frequency of a curved AlN pMUT with an average radius of 70 μm and radius of curvature of 1065 μm and AlN thickness of 2 μm operating in vacuum.

$${K}_{eff}^{2} = \frac{{f}_{a}^{2} - {f}_{r}^{2}}{{f}_{r}^{2}} \qquad ( 26 )$$

The maximum electromechanical coupling in vacuum that can be obtained with a circular piezoelectric diaphragm in this mode of operation is 5.5% and can be calculated as the following:

$${K}_{P}^{2} = \frac{2 {( {d}_{31} )}^{2} {Y}_{0}}{{ɛ}_{r} ( 1 - v )} \qquad ( 27 )$$

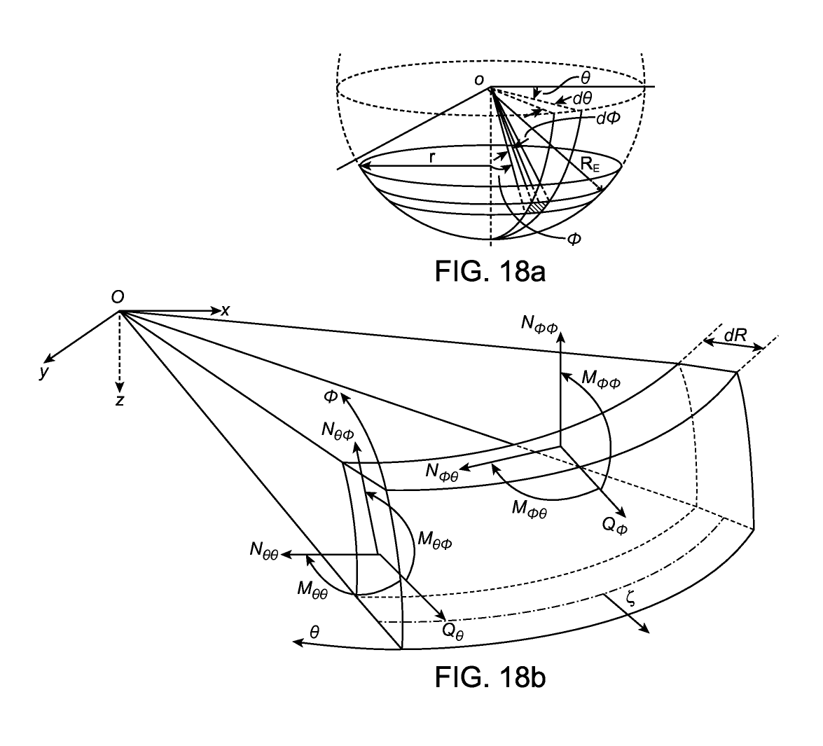

FIG. 22 shows a graph of the experimental impedance measurement for a curved pMUT with 120 μm in average diameter and 550 μm in radius of curvature which has a resonant frequency of 3.86 MHz, in air. The electromechanical coupling coefficient was calculated to be 2.1% from this figure which was 40× higher than a typical AlN based planar pMUT and was in the order of the planar electromechanical coupling factor which was the material limit. The 3.4% reduction was probably due to the parasitic capacitance around the diaphragm, in the concentrator.

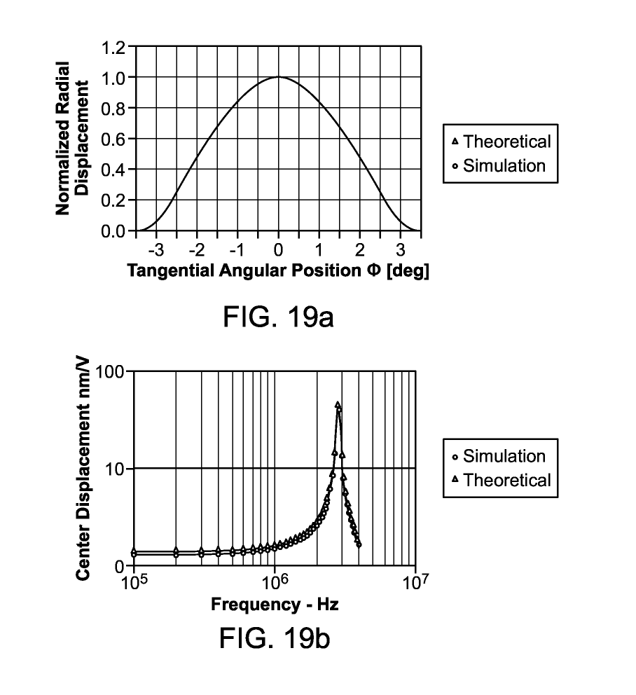

FIG. 23 a graph of center displacement (nm) vs. Vₚₚ (V), which demonstrates a linear response between center displacement and the input voltage from a curved pMUT operated at 500 kHz.

### Utility

The subject curved piezoelectric transducers, curved piezoelectric transducer arrays, devices that include the curved piezoelectric transducers, and methods of using the curved piezoelectric transducers find use in a variety of applications, such as applications where the conversion of energy into sound is desired. In some instances, the sound produced by the curved piezoelectric transducers is ultrasound. As such, the subject curved piezoelectric transducers find use in applications where the conversion of energy into ultrasound is desired.

Examples of applications where the conversion of energy into sound (e.g., ultrasound) is desired include medical applications where sound waves (e.g., ultrasound) are applied to a body tissue or fluid of a subject from a medical device that includes a subject curved piezoelectric transducer or array thereof. As such, the subject curved piezoelectric transducers, medical devices and methods find use in imaging a body tissue of a subject, such as ultrasound imaging a body tissue of a subject. For example, the subject curved piezoelectric transducers, medical devices and methods find use in ultrasound imaging of body tissues such as, but not limited to, brain tissue, muscle, bone, tendon, ligament, fat, blood vessel, skin, connective tissue, combinations thereof, and the like.

The subject curved piezoelectric transducers, medical devices and methods also find use in applications where the treatment of a body tissue of a subject with therapeutic sound waves (e.g., ultrasound) is desired. For example, the subject curved piezoelectric transducers, medical devices and methods find use in the treatment of a disease or condition in a subject in need of treatment of such disease or condition, such as, but not limited to, stroke, myocardial infarction, soft tissue injury, tendon injury, kidney stones, varicose veins, or cellulite. The subject curved piezoelectric transducers, medical devices and methods also find use in the treatment of a disease or condition in a subject in need of treatment of such disease or condition, such as, but not limited to, ligament sprain, muscle strain, tendonitis, joint inflammation, plantar fasciitis, metatarsalgia, facet irritation, impingement syndrome, bursitis, rheumatoid arthritis, osteoarthritis, scar tissue adhesion, gallstones, cataracts, or tumor.

The subject curved piezoelectric transducers, medical devices and methods also find use in applications where the measurement of a parameter of a body tissue or fluid of a subject using sound waves (e.g., ultrasound) is desired. For example, the subject curved piezoelectric transducers, medical devices and methods find use in measuring parameters such as, but not limited to, a thickness of the body tissue, a density of the body tissue or fluid, a velocity of a flow of the fluid, and the like, using sound waves (e.g., ultrasound).

Additional examples of applications where the conversion of energy into sound (e.g., ultrasound) is desired include fingerprint detection and body motion sensors, as well as various sensor devices as described herein. Thus, the subject curved piezoelectric transducers, devices and methods find use in applications where the detection of a target using sound (e.g., ultrasound) is desired. Other examples of applications where the conversion of energy into sound (e.g., ultrasound) is desired include ultrasonic transducer devices where ultrasound is applied to a target to modify the target. Thus, the subject curved piezoelectric transducers, devices and methods find use in applications where the modification of mechanical and/or physical properties of a target using ultrasound is desired. Further examples of applications where the conversion of energy into sound (e.g., ultrasound) is desired include data transmission via sound waves (e.g., ultrasound waves). Thus, the subject curved piezoelectric transducers, devices and methods find use in applications where the transmission of data via sound waves (e.g., ultrasound waves) is desired.

The subject curved piezoelectric transducers, devices and methods also find use in applications where a reduction in the energy and power consumption of an ultrasonic transducer device is desired. As described herein, the subject curved piezoelectric transducers have energy and power consumption requirements that may be orders of magnitude lower than typical planar pMUTs. The subject curved piezoelectric transducers, devices and methods also find use in applications where post-processing tuning, e.g., when curved pMUTs are used in an array configuration, is desired. The subject curved piezoelectric transducers, devices and methods also facilitate an increase in electromechanical coupling, and thus find use in applications where an increase in the efficiency of an ultrasonic transducer is desired. The subject curved piezoelectric transducers, devices and methods also facilitate an increase in responsivity, and thus find use in applications where a high response and sensitivity is desired.

Additional applications of the curved pMUT in a MUT Fingerprint Sensor System are described in more detail in U.S. Provisional Patent Application No. 61/846,925 filed Jul. 16, 2013, the disclosure of which is incorporated by reference herein. Additional applications of the curved pMUT in an In-Air Ultrasonic Rangefinding and Angle Estimation system are described in more detail in U.S. Provisional Patent Application No. 61/776,403 filed Mar. 11, 2013, the disclosure of which is incorporated by reference herein. In these sensor systems, the subject curved pMUTs may be used in place of the typical planar pMUTs in the system.

### Additional Embodiments

Aspects of the present disclosure include a curved piezoelectric micromachined ultrasonic transducers (pMUT).

In some embodiments, the curved pMUT has a radius of curvature from about 20 μm to 8,000 μm.

In some embodiments, the curved pMUT has a radius of curvature from about 100 μm to 2000 μm.

In some embodiments, the curved pMUT has a radius of curvature from about 600 μm to 1000 μm.

In some embodiments, the curved pMUT has an average diameter of from about 10 μm to 2 mm.

In some embodiments, the curved pMUT has an average diameter of from about 40 μm to 200 μm.

In some embodiments, the curved pMUT has an average diameter of from about 120-180 μm.

In some embodiments, the curved pMUT is tuned post processing,

In some embodiments, the curved pMUT is fabricated by complementary metal-oxide semiconductor (CMOS) compatible processing.

In some embodiments, the curved pMUT has an AC drive voltage of about 0.5V to 10V.

In some embodiments, the curved pMUT has an AC drive voltage of about 1V to 5V.

In some embodiments, the curved pMUT has an AC drive voltage of about 2V to 3V.

In some embodiments, the curved pMUT uses about 1-100 times less power than a planar pMUT of the same diameter.

In some embodiments, the curved pMUT uses about 10-50 times less power than a planar pMUT of the same diameter.

In some embodiments, the curved pMUT uses about 15-20 times less power than a planar pMUT of the same diameter.

In some embodiments, the curved pMUT has an electromechanical coupling of about 0.2% to 100%.

In some embodiments, the curved pMUT has an electromechanical coupling of about 10% to 60%.

In some embodiments, the curved pMUT has an electromechanical coupling of about 30% to 45%.

In some embodiments, the curved pMUT has a DC response from about 0.1 nm/V to 100.0 nm/V.

In some embodiments, the curved pMUT has a DC response from about 0.5 nm/V to 20.0 nm/V.

In some embodiments, the curved pMUT has a DC response from about 1 nm/V to 10 nm/V.

In some embodiments, the curved pMUT has a DC response 10-100 times that of a planar pMUT of the same average diameter.

In some embodiments, the curved pMUT has a DC response about 20-70 times that of a planar pMUT of the same average diameter.

In some embodiments, the curved pMUT has a DC response about 45-55 times that of a planar pMUT of the same average diameter.

In some embodiments, the curved pMUT has immunity to residual stress with aluminum nitride as a structural component of about 10 MPa to 500 MPa.

In some embodiments, the curved pMUT has immunity to residual stress with aluminum nitride as a structural component of about 50 MPa to 400 MPa.

In some embodiments, the curved pMUT has immunity to residual stress with aluminum nitride as a structural component of about 100 MPa to 300 MPa.

Aspects of the present disclosure include an array of the curved pMUT described herein.

In some embodiments, the curved pMUT array is provided in a personal electronic device.

In some embodiments, the curved pMUT array is provided in a personal electronic device within a fingerprint ID system or gesture recognition detector subunit.

In some embodiments, the curved pMUT array is provided in a personal electronic device fingerprint ID system and has an energy consumption per single fingerprint scan from about 1 μJ to 40 μJ.

In some embodiments, the curved pMUT array is provided in a fingerprint ID system and has an energy consumption per single fingerprint scan from about 5 μJ to 30 μJ.

In some embodiments, the curved pMUT array is provided in a fingerprint ID system and has an energy consumption per single fingerprint scan from about 10 μJ to 20 μJ.

Aspects of the present disclosure include a curved piezoelectric transducer that includes a substrate, a curved support layer comprising a peripheral portion in contact with the substrate, and a curved piezoelectric element disposed on the curved support layer.

In some embodiments, the substrate includes an opening through the substrate and a portion of the curved support layer is exposed through the opening.

In some embodiments, the curved support layer is suspended over the substrate by the peripheral portion.

In some embodiments, the curved piezoelectric transducer has a concave shape or a convex shape.

In some embodiments, the curved support layer is formed from a support layer having a central portion having residual stress and the peripheral portion, where the peripheral portion has residual stress.

In some embodiments, the central portion has residual tensile stress and the peripheral portion has residual compressive stress, or where the central portion has residual compressive stress and the peripheral portion has residual tensile stress.

In some embodiments, the central portion of the support layer includes a CMOS-compatible metal.

In some embodiments, the central portion of the support layer includes silicon nitride.

In some embodiments, the peripheral portion of the support layer includes an oxide.

In some embodiments, the peripheral portion of the support layer includes a low temperature oxide.

In some embodiments, the central portion of the support layer is circular.

In some embodiments, the peripheral portion of the support layer is annular and surrounds the periphery of the central portion.

In some embodiments, the curved piezoelectric element includes a first electrode layer, a piezoelectric layer, and a second electrode layer.

In some embodiments, the curved piezoelectric transducer has a radius of curvature ranging from 10 μm to 10,000 μm.

In some embodiments, the curved piezoelectric transducer has a diameter ranging from 10 μm to 5 mm.

In some embodiments, the curved piezoelectric transducer has an electromechanical coupling ranging from 10% to 100%.

In some embodiments, the curved piezoelectric transducer has a DC response ranging from 0.1 nm/V to 100 nm/V.

In some embodiments, the curved piezoelectric transducer has a resistance to residual stress ranging from 10 MPa to 500 MPa.

Aspects of the present disclosure include a device having a substrate, and an array of curved piezoelectric transducers on the substrate, where each curved piezoelectric transducer includes a curved support layer having a peripheral portion in contact with the substrate, and a curved piezoelectric element disposed on the curved support layer.

In some embodiments, the array includes 10 or more curved piezoelectric transducers.

Aspects of the present disclosure include a method of making a curved piezoelectric transducer. The method includes producing a curved piezoelectric element on a curved support layer on a first surface of a substrate, where the curved support layer includes a peripheral portion in contact with the first surface of the substrate.

In some embodiments, the method includes forming a curved depression in the first surface of the substrate prior to the producing.

In some embodiments, the producing includes depositing the support layer in a curved depression in the first surface of the substrate, and depositing the piezoelectric element on the support layer.

In some embodiments, the method further includes removing substrate material from an opposing second surface of the substrate to produce a opening through the substrate to expose a portion of the curved support layer.

In some embodiments, the removing includes etching the opening through the substrate.

In some embodiments, the producing includes a chemical or physical deposition process.

In some embodiments, the producing includes depositing a support layer on the first surface of the substrate, where the support layer includes a central portion having residual tensile stress and the peripheral portion, where the peripheral portion has residual compressive stress, removing substrate material from an opposing second surface of the substrate to produce a opening through the substrate to expose a portion of the support layer, and depositing the piezoelectric element on the support layer.

In some embodiments, depositing the piezoelectric element includes depositing a first electrode layer on the support layer, depositing a piezoelectric layer on the first electrode layer, and depositing a second electrode layer on the piezoelectric layer.

In some embodiments, the method further includes forming a first electrical contact to the first electrode layer and a second electrical contact to the second electrode layer.

As can be appreciated from the disclosure provided above, embodiments of the present invention have a wide variety of applications. Accordingly, the examples presented herein are offered for illustration purposes and are not intended to be construed as a limitation on the invention in any way. Those of ordinary skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Examples

FIG. 4a and FIG. 4b show displacement versus frequency plots of a curved pMUT having a 190 μm diameter and 1065 μm radius of curvature, both by computer simulation and experiment on fabricated devices. Acoustic-piezoelectric frequency domain simulations were conducted in COMSOL Multiphysics to estimate the frequency response of the diaphragms. FIG. 4a shows the center displacement (nm/V) versus frequency (MHz) plot for a curved aluminum nitride (AlN) pMUT with a total thickness of 2.5 μm (including the metal electrode and bottom oxide support layer), 190 μm average diameter, and 1065 μm radius of curvature, operated in air. As shown in FIG. 4a, the simulated center DC displacement was 1.4 nm/V and the displacement at its resonance was 75 nm/V at 2.45 MHz.

FIG. 4b shows a graph of measurements conducted using a Laser Doppler Vibrometer (LDV) in air of a device having a 190 μm average diameter and 1065 μm radius of curvature. The measured resonant frequency was 2.19 MHz, with a 1.1 nm/V DC vertical displacement, and 45 nm/V center displacement at resonance. It is noted that LDV can only measure the velocity/displacement when the target is under motion, such that the DC displacement was measured at very low frequencies away from the resonant frequency.

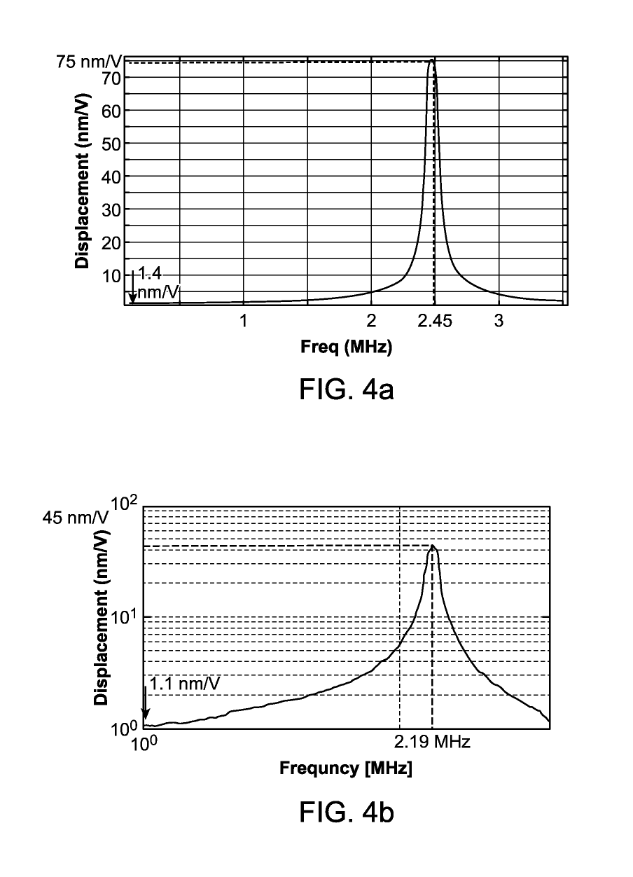

FIG. 5 is a scanning electron microscopy (SEM) image providing a cross-section view of a curved pMUT device according to embodiments of the present disclosure. FIG. 5 shows a 2D image, similar to that shown in FIG. 2. Clamp parts 12 are shown at the edge of curved pMUT membrane 1 at the boundary condition, which was thus clamped or anchored. In this case, membrane 1 was the part of the sphere between the clamped circle and the backside hole 8. The backside hole 8 was fabricated with deep reactive ion etching to release the membrane, e.g., the bottom surface of the membrane 1 was exposed through the backside hole 8. As shown in FIG. 5, backside hole 8 was the columnar-looking area below the bright hemisphere, which was part of the sphere of physical feature of curvature 4. Backside hole 8 was the etched out area under the sphere. Backside hole 8 released the membrane from the backside silicon wafer 2, which allowed the membrane to vibrate during use.

Not specifically shown in FIG. 5, but provided for context, the second molybdenum layer 20, served functionally as the top electrode, and the first molybdenum layer 18, served functionally as the bottom electrode. Aluminum nitride layer 22 was both the structural layer and the piezoelectric layer. Thus, voltage was applied at aluminum nitride layer 22 which converted the electrical energy to mechanical energy and also was the main structural electromechanical layer.

A silicon oxide layer on the bottom of pMUT membrane 1 served as the stop layer for the deep reactive ion etching. When etching from the backside, the plasma etched silicon 6, but stopped at the silicon oxide layer. In this manner, the whole pMUT membrane 1 was protected during the etching process to produce the backside hole 8.

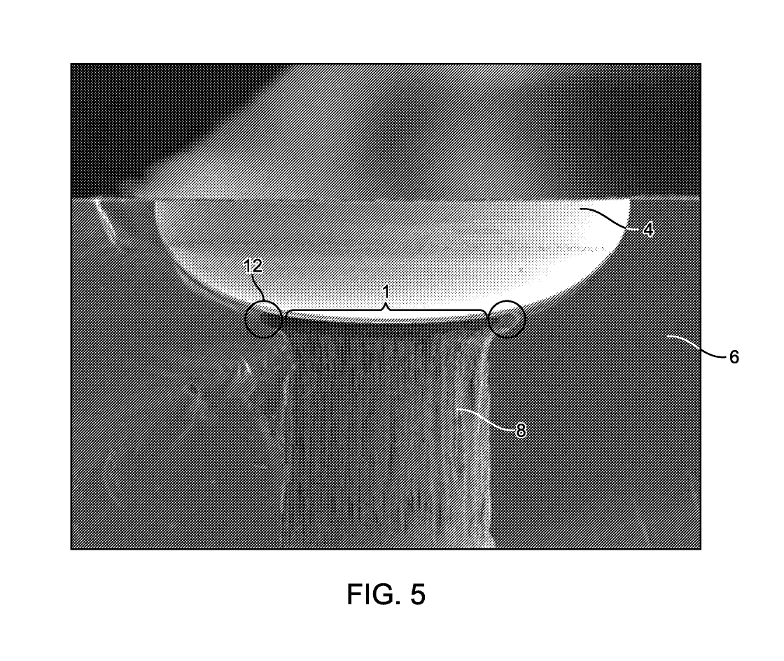

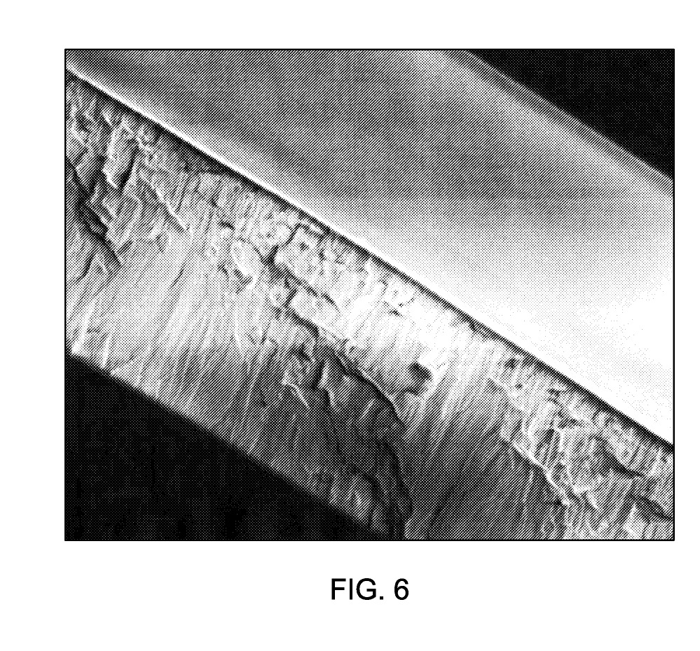

FIG. 6 is a high focus scanning electron microscopy (SEM) image providing a cross-section view of aluminum nitride layer 22, which has been sputtered onto the physical feature of curvature 4 and thus is a component of the curved pMUT membrane 1. A feature shown in FIG. 6 is the polarization of the aluminum nitride layer 22. FIG. 6 shows that the polarization direction of the AlN crystalline structure was perpendicular to the curvature of the diaphragm. The crescent orientation was such that the aluminum nitride layer 22 material was polarized perpendicular to the curve of the curved pMUT. When aluminum nitride was sputtered on the curved areas, the aluminum nitride crescent orientation, or polarization, was fabricated to be perpendicular to the curve. The crescent orientation shown in FIG. 6 and FIG. 7 shows that the polarization was in the radial direction, which means that the polarization of the piezoelectric layer was perpendicular to the curvature of the curved pMUT.

FIG. 7 is an SEM image providing a cross-section view of aluminum nitride layer 22 which was sputtered onto the physical feature of curvature 4 and thus was a component of the curved pMUT membrane 1. This SEM picture in FIG. 7 is shown at a lower magnification than that in FIG. 6. The arrows superimposed on the SEM picture of FIG. 7 show the polarization direction on different parts of the surface. Note that the polarization direction was perpendicular to the curvature. As such, the polarization direction was defined in the computer simulations as being perpendicular to the curvature. Aluminum nitride was particularly susceptible to this sputtered polarization that occurred during its deposition.

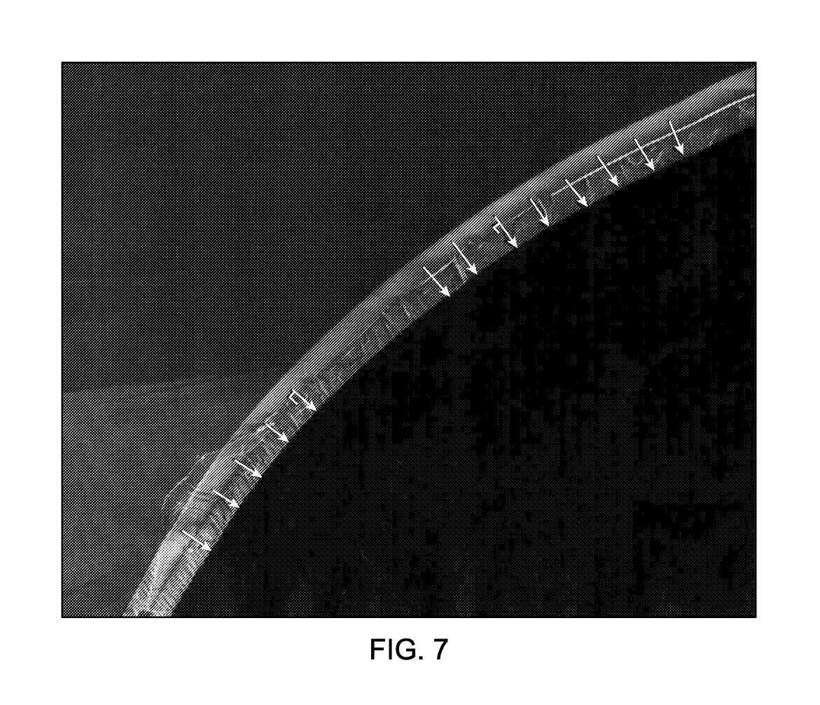

FIG. 8 shows computer simulation results for the curved pMUT's total displacement. This simulation was performed to determine the effect of the curvature of the structure and included a finite element analysis using COMSOL Multiphysics, which is a finite element method (FEM) tool. FIG. 8 shows DC displacement versus 1V. The computer simulation indicated a maximum displacement of 1.28 nm. In practice, embodiments of the curved pMUT have a maximum displacement of 1.1 nm, which demonstrates a reasonably close correlation (within 15%) between the computer simulation and the function of the curved pMUT in practice. Similarly, there is close correlation between the computer simulation and the device shown in FIG. 4a, which had a displacement of 1.1 nm/V in low frequencies in DC mode.

In the computer simulation, the center of the membrane was more deflected than the edge of the membrane. The clamped area of the membrane was indicated as not moving at all, which was appropriate to the system. In this simulation, the edge of the circular 3-dimensional figure represented the clamped areas, the center represented the membrane 1, and the deflection was demonstrated in this simulation.

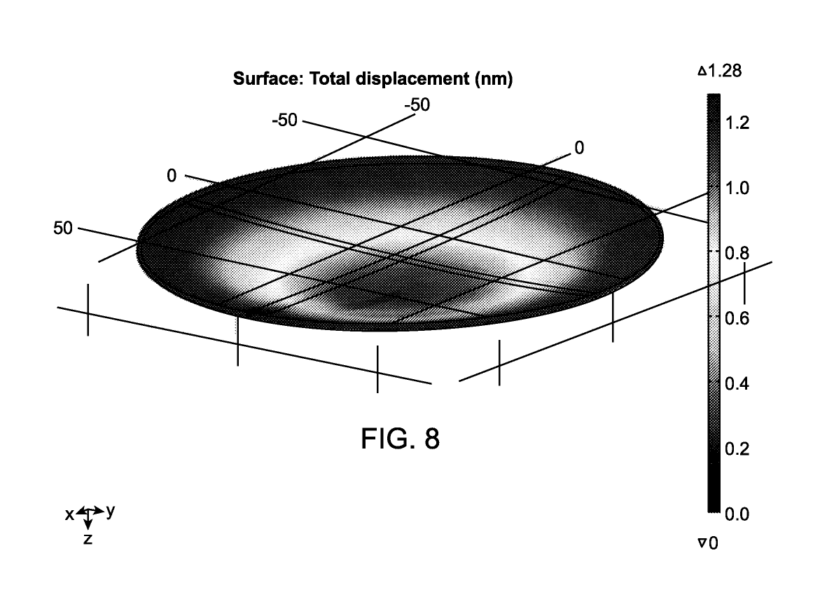

FIG. 9a and FIG. 9b show schematic drawings of cross-sections of curved pMUT membrane 1 with different curvature configurations. Curved pMUTs of the present disclosure may have a double curvature, such as a concave-convex curvature (FIG. 9b; e.g., if full movement is desired), or a single curvature (FIG. 9a), where the curved pMUT membrane 1 is constrained at both ends. FIG. 9a is an example of a curved pMUT having a single curvature, where clamp parts 12 substantially reduce or prevent rotation at that point on the curved pMUT membrane 1. During activation, curved pMUT membrane 1 may be constrained from moving at the clamp regions. FIG. 9b shows an example of a curved pMUT having a double curvature of the pMUT membrane 1. Inflection point 24 is provided in pMUT membrane 1. The inflection point 24 may be positioned on the pMUT membrane 1 as a certain percentage of the radius. When a DC bias is applied, the pMUT membrane 1 in pMUT membrane section 26 (i.e., concave curvature) can move to the position indicated by the dotted lines, as it is substantially unrestrained. Similarly, pMUT membrane section 28 (i.e., convex curvature) can deflect to accommodate the movement of the membrane.

As a result of the increased freedom of movement provided by the double curvature of pMUT membrane 1 shown in FIG. 9b, the curved pMUT may displace more volume than the single curvature example in FIG. 9a. As described above, pMUT membrane sections 26 and 28 are substantially unrestrained, so they can move together during use. Inflection points such as inflection points 24 may decrease the anchor loss. The double curvature design may provide more degrees of freedom to the boundary conditions. As a result, the movement of curved pMUT may be substantially unrestricted, thus decreasing the anchor losses and increasing the freedom of movement, which in turn may result in higher efficiency, more displacement, and higher electromechanical coupling. As used herein “anchor losses” refer to losses of input energy. In function, as the membrane starts to move, it is prone to rotation. This rotation may be limited due to areas 12 where it is clamped as shown in FIG. 9a. However, in FIG. 9b, inflection points 24 serve functionally as hinges, allowing pMUT membrane sections 26 and 28 to deflect more because of the added degrees of freedom.

FIG. 11 shows a comparison between simulation and experimental results. The graph in FIG. 11 shows the change of resonant frequency of the DC displacement in terms of the radius of curvature. To determine the effect of the curvature of the structure, both an analytical model as shown in FIG. 11, and finite element analyses using COMSOL Multiphysics as shown in FIG. 8 were tested and showed good consistency.

The solid curve in FIG. 11 is the predicted DC displacement of a curved pMUT with 2 μm-thick aluminum nitride, 140 μm in average diameter with respect to different radii of curvature. The DC displacement increased as the radius of curvature increased; reached a maximum point; and decreased with further increase of the curvature. A curved pMUT thus had a higher resonant frequency than a flat pMUT with the same average diameter. As a result the curved pMUT had a higher volumetric velocity (the product of higher frequency and higher displacement) than a planar pMUT to generate a higher acoustic pressure.

The symbols in FIG. 11 are the experimental data measured from devices with different radii of curvature. The data from a planar pMUT was from a similar process run with 1 μm-thick aluminum nitride, 3 μm-thick silicon as the structural layer and 70% top electrode coverage. The experimental results for curved pMUTs were consistent with the simulation results both in terms of the center displacement and resonant frequency. More than one order of magnitude higher DC displacement was achieved from the curved pMUT as compared with the planar pMUT.

From the comparison, the experimental data generally followed the trend of the simulation. But both the resonant frequency and the displacement were somehow lower than the simulation results. This difference may be due to residual stresses in the aluminum nitride, which were about 300 megapascals. For example, the curved pMUTs were more immune, that is, less sensitive to residual stress than typical planar pMUTs. Thus, the curved pMUT will function when flat pMUTs will not. For instance, a curved pMUT subject to residual stress can release the stress. But in a planar pMUT, there is no room for release. Analogous to a guitar string, the curved pMUT may deflect less with more tension. As a result, a curved pMUT may have significant immunity to residual stress. For instance, if there is residual stress, the initial deflection would change, changing the radius of curvature of the curved pMUT. These effects may change the frequency, but may not significantly affect the function of the device.

### Self-Curved Piezoelectric Transducers

A process to make self-curved diaphragms by engineering residual stress in thin films was developed to construct highly responsive piezoelectric micromachined ultrasonic transducers (pMUT). This process enabled high device fill-factor for better than 95% area utilization with controlled formation of curved membranes. The placement of a 0.65 μm-thick, low stress silicon nitride (SiN) film with 650 MPa of tensile residual stress and a low temperature oxide (LTO) film with 180 MPa of compressive stress sitting on top of a 4 μm-thick silicon film resulted in the self-curved diaphragms. A curved pMUT with 200 μm in nominal radius, 2 μm thick aluminum nitride (AlN) piezoelectric layer, and 50% SiN coverage has resulted in a 2.7 μm deflection at the center and resonance at 647 kHz. Low frequency and resonant deformation responses of 0.58 nm/V and 40 nm/V at the center of the diaphragm were measured, respectively.

This process enabled foundry-compatible CMOS process and large fill-factor for pMUT applications.

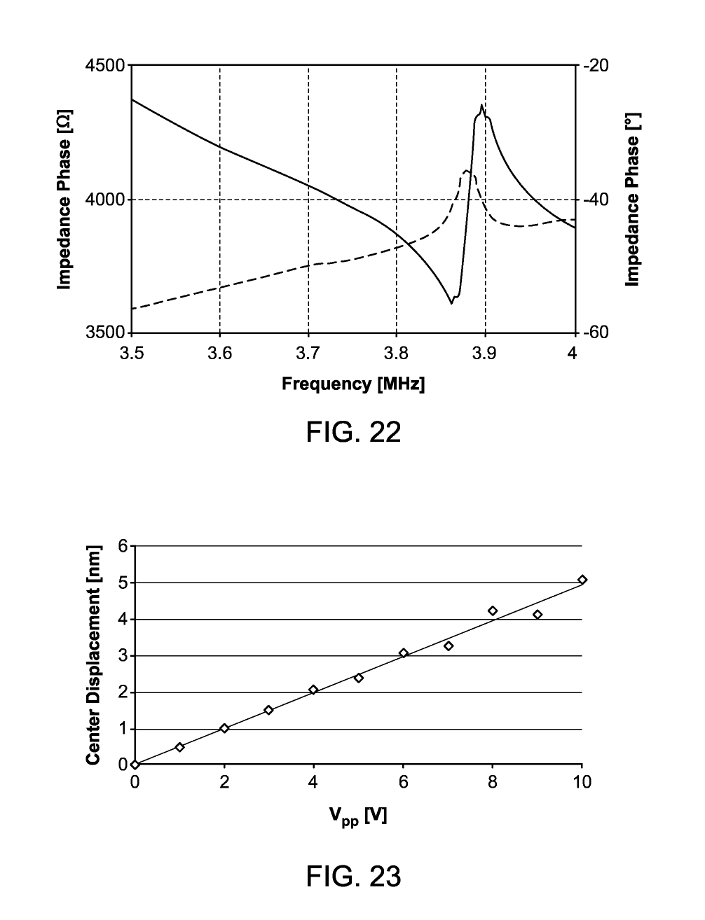

FIG. 26 shows a 3D schematic diagram of the stress engineered, self-curved pMUT 2600. The curved structure was produced by a piezoelectric AlN layer 2610 sandwiched between a bottom metal electrode 2620 and a top metal electrode 2630 on top of a silicon diaphragm 2640 above a sub-layer of an oxide 2690, with a self-generated curvature due to residual stresses in the films. The silicon nitride layer 2650 and silicon oxide layer 2660 with known tensile and compressive stress, respectively, were introduced on top of the device layer on a SOI wafer 2670 to induce the targeted concave-shape structure. A via 2680 through the top electrode and piezoelectric layer allows an electrical contact to be made to the bottom electrode. The final curvature of the diaphragm was caused by the balance of stresses in various thin films and can be adjusted by the size and properties of the thin films. Suspended diaphragms bend downward as illustrated without unutilized portions as those fabricated previously by the wet etching process. As such, high fill factor was achieved.

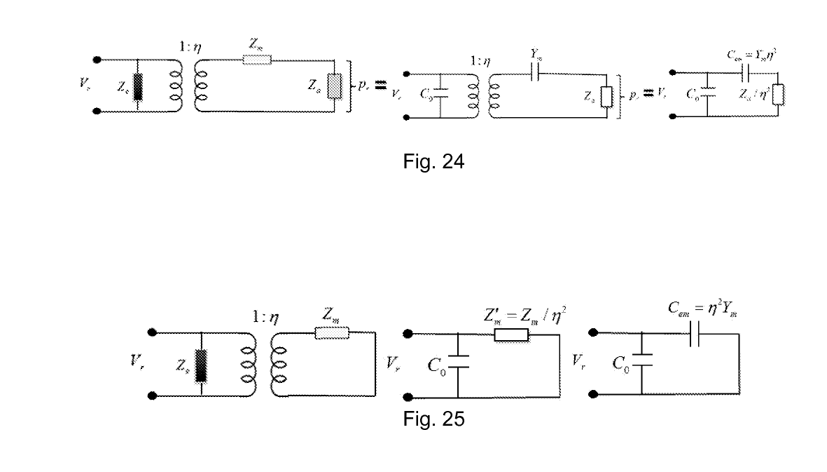

The cross-sectional diagram in FIG. 27 (top) shows the stress engineered curved pMUT. The combination of tensile stressed silicon nitride layer 2710 (partially covering the central region of the circular diaphragm) and the compressive stressed LTO 2720 (covering the rest of the diaphragm) resulted in the concave-shape structure. Analytically, if a flat, stress-free, clamped diaphragm is deflected downward, radial tensile stress 2730 is formed at the outer portion and radial compressive stress is established at the inner portion of the top surface of the diaphragm. The stress neutral line (zero stress) or the inflection circle was located at about 0.65r position, where r is the radius of the diaphragm. Therefore, the stress-free concave-shape diaphragm as illustrated in FIG. 27 (top) was produced by depositing a thin film with tensile residual stress 2710 at the inner portion and a thin film with residual compressive stress 2720 on the outer portion of a silicon support layer 2760. The silicon support layer was disposed on a substrate 2770, with a layer of oxide 2780 between the substrate and the support layer. Before release of the residual stress, the diaphragm was substantially flat as shown by dotted lines 2750. Once the residual stresses were released, a curved downward diaphragm was self-constructed due to a bending moment 2740 produced by the interaction of the residual tensile and compressive stresses. The curvature of the self-curved diaphragm was configured to achieve a desired center deflection, g, by tuning the silicon nitride and oxide residual stresses σ_{SiN} & σ_{Ox}, the silicon nitride and oxide thickness h_{SiN} & h_{Ox}, their distances from neutral axes Z_{SiN} & Z_{Ox}, Poisson's ratios ν_{SiN} & ν_{Ox}, and the coverage radius r_{N}. The radial force per unit length due to the residual stress in the nitride film was simplified as σ_{SiN}h_{SiN}, and the moment per unit length generated by the nitride layer about the neutral axis of the diaphragm stack was σ_{SiN}h_{SiN}Z_{SiN}. The residual stresses in the thin films generated high moments to bend the released diaphragm after the backside silicon was etched away:

$${W}_{s} ( r ) = \frac{{πσ}_{SiN} {h}_{SiN} {Z}_{SiN} }{rD ( 1 - {v}_{SiN} )} {\sum }_{k} \frac{{O}_{k} ( {r}_{N} )}{{Λ}_{k} {Γ}_{k}} {Ψ}_{k} ( r ) - \frac{{πσ}_{Ox} {h}_{Ox} {z}_{Ox}}{rD ( 1 - {v}_{Ox} )} {\sum }_{k} \frac{{o}_{k} ( {r}_{N} )}{{Λ}_{k} {Γ}_{k}} {Ψ}_{k} ( r )$$

where r and D are the diaphragm nominal radius and flexural rigidity, respectively and Oₖ, Ψₖ, Λₖ and Γₖ are functions defined in the above equations. By adding the bottom and top electrodes 2785 and the piezoelectric AlN layer 2790 to complete the fabrication process after FIG. 27 (top), the stress engineered curved pMUT operated as shown in FIG. 27 (bottom) in the transmission mode under an AC voltage. The induced stress in the piezoelectric layer due to the d₃₁ effect stretched and compressed the diaphragm, such that it resonated in the flexural mode to emit acoustic waves. The induced stress due to d₃₁ had a vertical component in the desired vertical motion to enhance electromechanical coupling of the device.

### Fabrication

### Process Flow

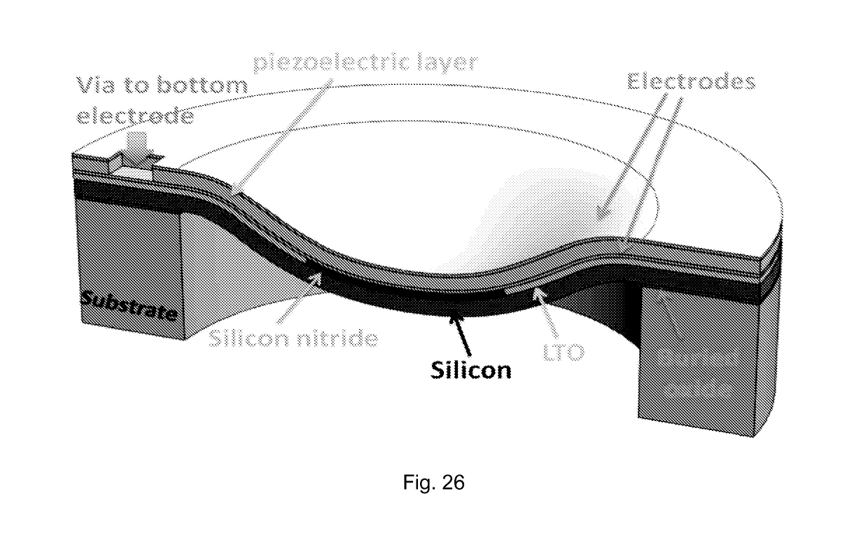

FIG. 28 shows the process flow of the stress engineered self-curved pMUT. The process started with the deposition and pattering of a 650 nm-thick silicon nitride layer with naturally inherent tensile residual stress (650 MPa) on a SOI wafer with a 4 μm-thick device layer and a 1 μm-thick BOX layer (FIG. 28, panel a). The next step was LTO deposition followed by chemical mechanical polishing (CMP) (FIG. 28, panel b). There were two purposes for LTO deposition and CMP: (1) to smoothen out the surfaces for the future Mo/AlN/Mo sputtering on the diaphragm area, and (2) to further help the curvature formation by using the LTO residual compressive stress, which in this case was 180 MPa (compressive). Backside deep reactive ion etching (DRIE) was then used to release the self-curved diaphragm (FIG. 28, panel c). After the BOX layer under the diaphragm was removed, the diaphragm bent in a concave form due to the residual stresses of the nitride and oxide thin films before the depositions of electrode layers and AlN piezoelectric layer using active sputtering of Mo/AlN/Mo as bottom electrode, piezoelectric layer, and top electrode with 150 nm, 2 μm, and 150 nm in thickness, respectively. The via to the bottom electrode was opened using a combination of dry and wet AlN etching steps by chlorine based plasma and the MF-319 developer, respectively (FIG. 28, panel d) in order to reduce the damage to the Mo bottom electrode layer. Top Mo was patterned beforehand using SF6 plasma etching.

### Fabrication Results

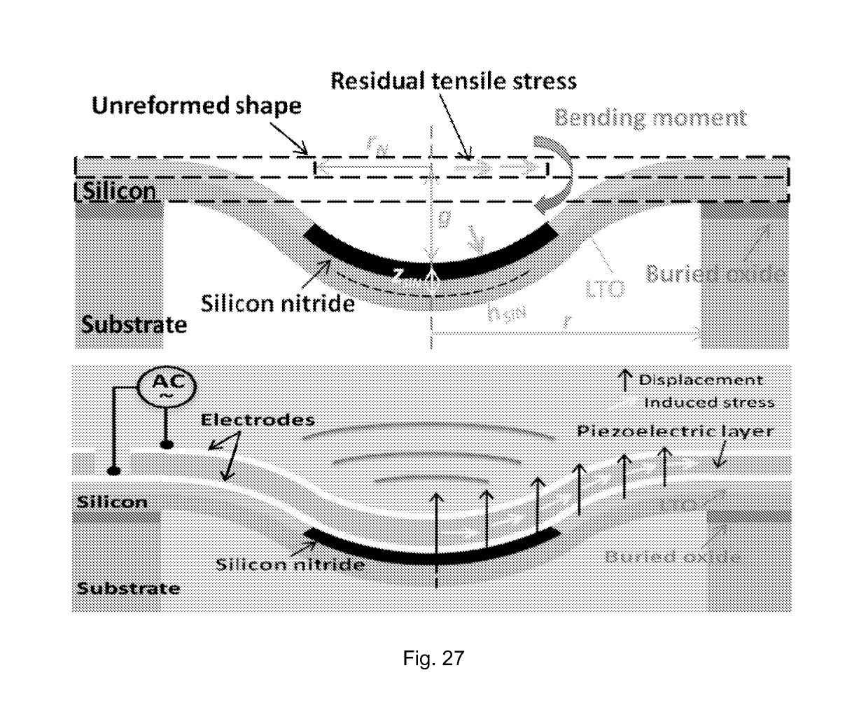

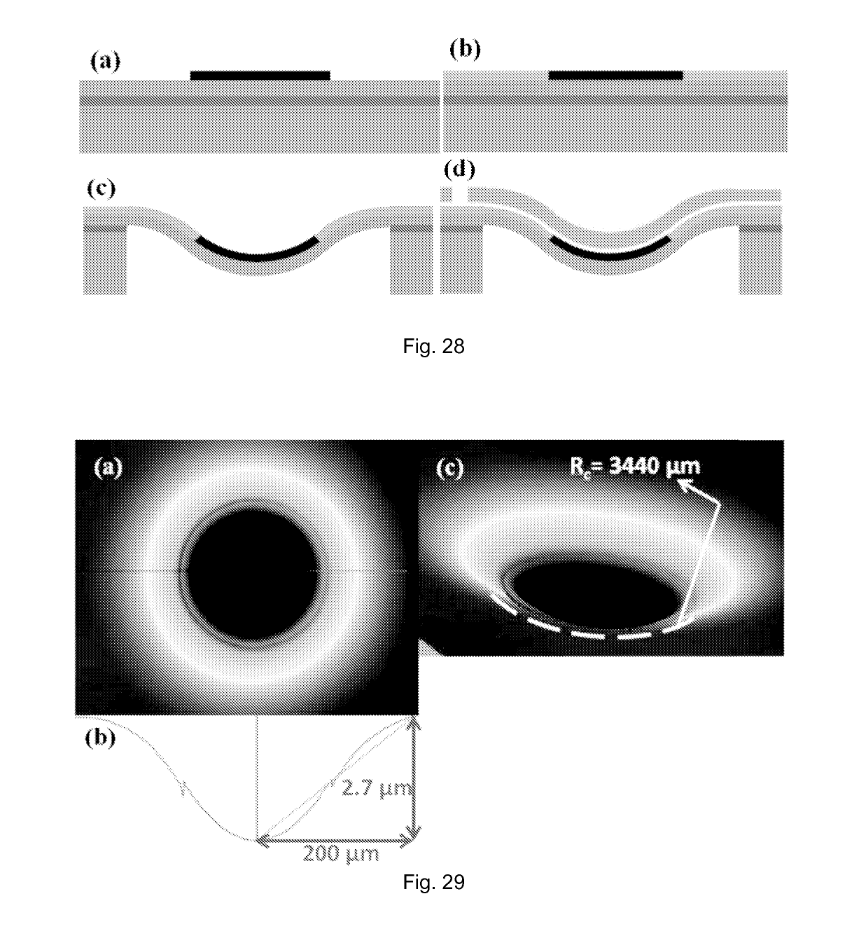

FIG. 29 shows confocal laser scanned images captured using Olympus LEXT OLS4000 3D Confocal Laser Microscope of a fabricated, self-curved pMUT with 400 μm in diameter, 50% nitride coverage, and measured center deflection of 2.7 μm. FIG. 30, panel a, and FIG. 30, panel b, are SEM micrographs of two self-curved pMUTs showing the clamped and curved diaphragm. FIG. 30, panel c, shows a cross-sectional view of the diaphragm stack composed of, from bottom to top, the buried oxide, silicon device, silicon nitride, and LTO layers as well as the Mo bottom electrode, AlN layer, and the top Mo electrode, respectively. FIG. 30, panel d, is an enlarged view on the AlN illustrating good crystal orientation.

### RESULTS AND DISCUSSION

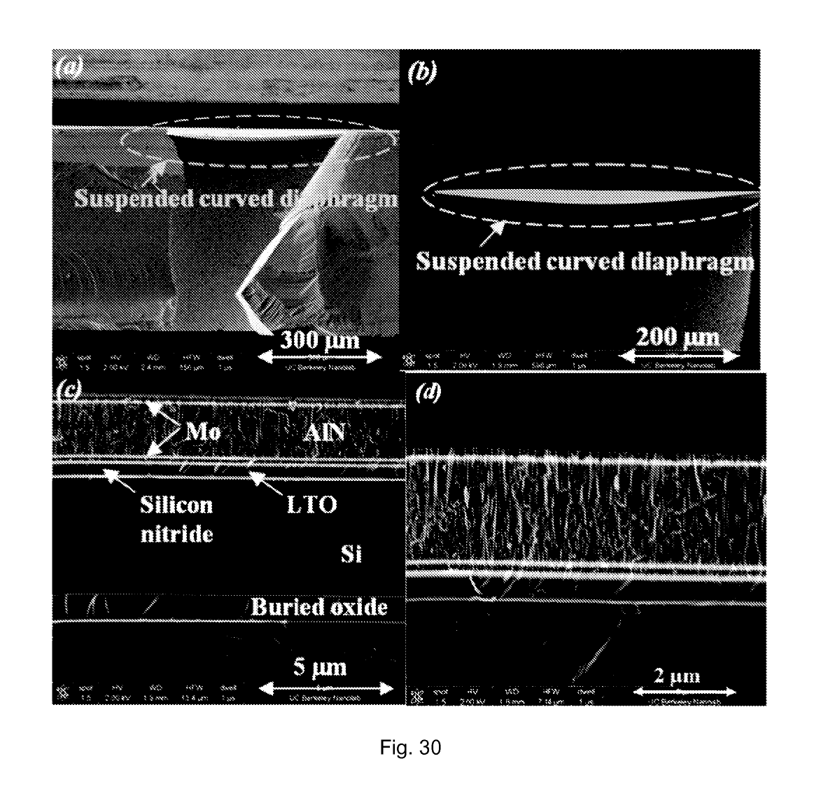

The center diaphragm deflection, g, versus the silicon nitride radial coverage percentage, r_{N}, is shown in FIG. 31 for a diaphragm with average radius of 200 μm and silicon thickness of 4 μm. The 650 nm-thick SiN had a tensile residual stress of 650 MPa and the LTO had a compressive residual stress of 180 MPa. Results showed good consistency between theory (coded in Matlab™), simulation (COMSOL), and experimental data. It was observed that the higher nitride coverage resulted in higher center deflection for the range of nitride coverages between 40%-55%. Since the curvature of the diaphragm affected both the resonant frequency and the excited deformation of the devices, the SiN radial coverage ratio was used in the design process to optimize the device performance. If the coverage percentage increased to be above the inflection circle (roughly 65%-70% of the radius of the diaphragm), the center deflection reduced as compressive regions of the diaphragm reduced the bending moment. The optimal design values were analyzed or simulated with known properties and parameters of the thin films.

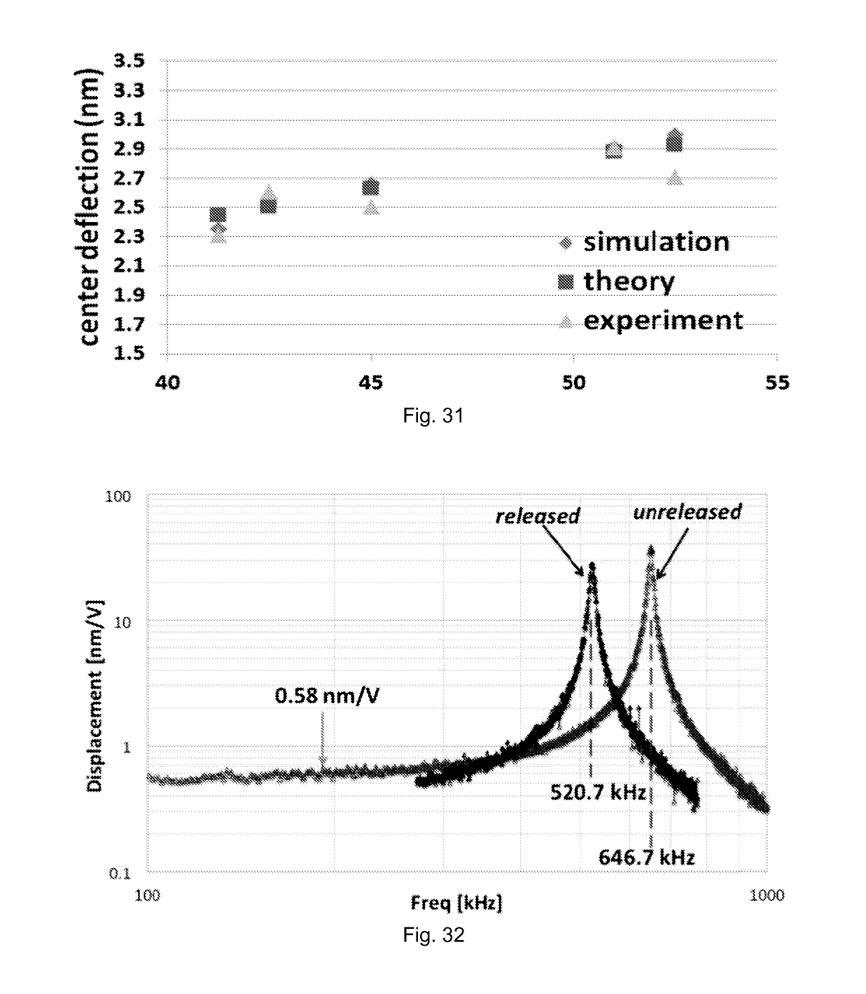

The dynamic responses of a fabricated curved pMUT without (released) and with (unreleased) the bottom silicon layer were measured using Laser Doppler Vibrometer (LDV) and presented in FIG. 32. Resonant frequency reduced from 646.7 kHz to 520 kHz while low frequency displacement remained at 0.58 nm/V after the removal of the silicon layer. It was expected from Finite Element Modeling (FEM) that the released diaphragm without silicon would have lower resonant frequency of 381 kHz and higher low-frequency displacement of 8.5 nm/V as compared to the measured values. The discrepancy between the theoretical and experimental data may be attributed to the excessive residual stress in the as-deposited AlN layer (tensile 170 MPa).

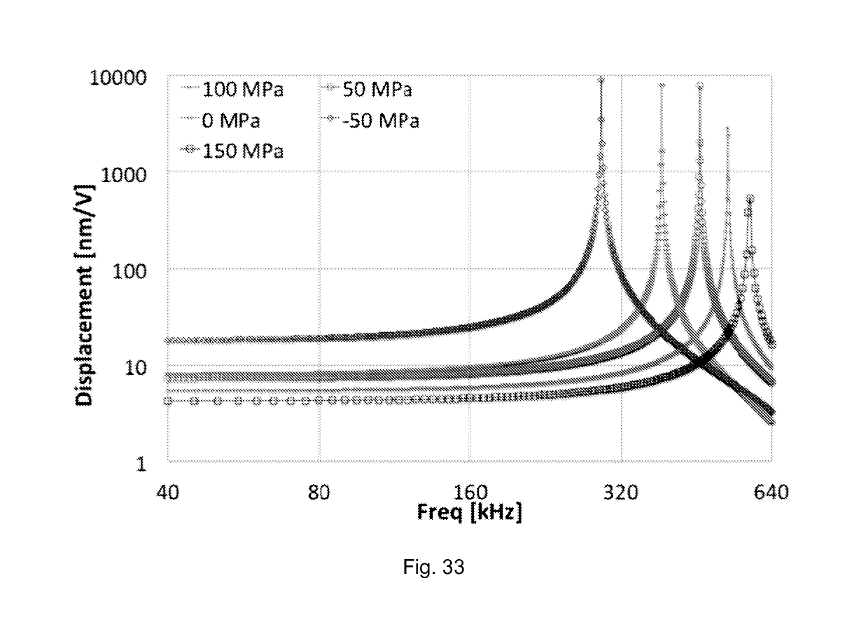

FIG. 33 shows the effects of residual stress in AlN on the dynamic responses of stress engineered curved pMUT devices. As the residual stress in the AlN increased, the low-frequency displacement per unit input voltage decreased and the resonant frequency increased. It was expected that the device performance would match with the simulated values when the stress in the sputtered AlN was controlled to be within 30 MPa.

It is to be understood that this invention is not limited to particular aspects or aspects described, as such may vary. It is also to be understood that the terminology used herein is for the purpose of describing particular aspects only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It will be appreciated that as used herein, the term “dissolve” may be used to indicate melt, soften, liquefy, thaw, disrupt, break up, break open, break apart, or otherwise destroy a layer or coating of material encapsulating an ingestible event marker either wholly or partially to release the ingestible event marker.

As will be apparent to those of skill in the art upon reading this disclosure, each of the individual aspects described and illustrated herein has discrete components and features which may be readily separated from or combined with the features of any of the other several aspects without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and aspects of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary aspects shown and described herein. Rather, the scope and spirit of present invention is embodied by the appended claims.

1. A medical device configured to direct sound waves to a body tissue of a subject, the medical device comprising:
a housing; and
a curved piezoelectric transducer comprising:
a substrate;
a curved support layer comprising a peripheral portion in contact with the substrate; and
a curved piezoelectric element disposed on the curved support layer,
wherein the curved piezoelectric transducer is configured to direct sound waves produced by the curved piezoelectric transducer to the body tissue of the subject.

2. The medical device of claim 1, further comprising a processor connected to the curved piezoelectric transducer.

3. The medical device of claim 2, further comprising a sound wave detector connected to the processor.

4. The medical device of claim 2, further comprising a wireless communication device connected to the processor.

5. The medical device of claim 1, wherein the medical device is selected from the group consisting of an imaging device, a therapeutic device, and a measurement device.

6. The medical device of claim 1, wherein the sound waves are ultrasound waves.

7. The medical device of claim 1, wherein the curved piezoelectric element comprises:
a first electrode layer;
a piezoelectric layer; and
a second electrode layer.

8. A method of directing sound waves to a body tissue of a subject, the method comprising:
producing sound waves from a medical device comprising:
a housing; and
a curved piezoelectric transducer comprising:
a substrate;
a curved support layer comprising a peripheral portion in contact with the substrate; and
a curved piezoelectric element disposed on the curved support layer,
wherein the curved piezoelectric transducer is configured to direct the produced sound waves to the body tissue of the subject.

9. The method of claim 8, wherein the method is a method of imaging the body tissue of the subject.

10. The method of claim 9, wherein the method comprises:
applying the produced sound waves to the body tissue from the curved piezoelectric transducer;
detecting sound waves reflected from the body tissue; and
producing an image of the body tissue from the detected sound waves.

11. The method of claim 10, wherein the body tissue is selected from the group consisting of brain tissue, muscle, bone, tendon, ligament, fat, blood vessel, skin, and connective tissue.

12. The method of claim 8, wherein the method is a method of treating the body tissue of the subject with therapeutic sound waves.

13. The method of claim 12, wherein the method comprises applying the produced sound waves to the body tissue of the subject from the curved piezoelectric transducer to treat the subject for a condition in need of treatment.

14. The method of claim 13, wherein the condition is selected from the group consisting of stroke, myocardial infarction, soft tissue injury, tendon injury, kidney stones, varicose veins, and cellulite.

15. The method of claim 13, wherein the condition is selected from the group consisting of ligament sprain, muscle strain, tendonitis, joint inflammation, plantar fasciitis, metatarsalgia, facet irritation, impingement syndrome, bursitis, rheumatoid arthritis, osteoarthritis, scar tissue adhesion, gallstones, cataracts, and tumor.

16. The method of claim 8, wherein the method is a method of measuring a parameter of a body tissue or fluid of the subject.

17. The method of claim 16, wherein the method comprises applying the produced sound waves to the body tissue or fluid from the curved piezoelectric transducer to measure the parameter of the body tissue or fluid.

18. The method of claim 17, wherein the parameter is a thickness of the body tissue, a density of the body tissue or fluid, or a velocity of a flow of the fluid.

19. The method claim 8, wherein the sound waves are ultrasound waves.

20. The method of claim 8, wherein the curved piezoelectric element comprises:
a first electrode layer;
a piezoelectric layer; and
a second electrode layer.
